SEQUENCE LISTING (47 sequences)

SEQ ID NO: 1 - DNA, 1432 bp, Alistipes
  misc_feature: Alistipes sp. HGB5
ggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacagctctatgagttgtaaactgcttttgtactagggtaaacgctcctacgtgtaggagtctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgtgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctcctttct

SEQ ID NO: 2 - DNA, 1436 bp, Alistipes
  misc_feature: Alistipes sp. HGB5
ggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacagctctatgagttgtaaactgcttttgtactagggtaaacgctcctacgtgtaggagtctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgtgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctcctttctggag

SEQ ID NO: 3 - DNA, 1424 bp, Atopobium parvulum
  misc_feature: Atopobium parvulum type strain (IPP 1246)
gatgaacgctggcggcgcgcctaacacatgcaagtcgaacgattaaagcaccttcgggtgtgtataaagtggcgaacggctgagtaacacgtgggcaacctgcccctttcattgggatagccacgggaaaccgtggataataccgaatacttcgagacttccgcatggaagactcgagaaagctccggcggagagggatgggcccgcggcctgttagcttgttggtggggtaacggcctaccaaggcaatgatgggtagctgggttgagagaccgaccagccagattgggactgagacacggcccagactcctacgggaggcagcagtggggaatcttgcacaatgggcgaaagcctgatgcagcgacgccgcgtgcgggatgaaggccttcgggttgtaaaccgctttcagcagggacgaggcgaaagtgacggtacctgcagaagaagccccggctaactacgtgccagcagccgcggtaatacgtagggggcaagcgttatccggattcattgggcgtaaagcgctcgtaggcggtctgttaggtcgggagttaaatccgggggctcaacccccgctcgctctcgataccggcagacttgagtttggtaggggaaggtggaattcctagtgtagcggtggaatgcgcagatattaggaagaacaccagtggcgaaggcggccttctgggccataactgacgctgaggagcgaaagctaggggagcaaacaggattagataccctggtagtcctagccgtaaacgatggacactaggtgtgggggagtatttcttccgtgccgcagctaacgcattaagtgtcccgcctggggagtacggccgcaaggctaaaactcaaaggaattgacgggggcccgcacaagcagcggagcatgtggtttaattcgaagcaacgcgaagaaccttaccagggcttgacatttaggtgaagcggcggaaacgtcgtggccgaaaggagcctaaacaggtggtgcatggctgtcgtcagctcgtgtcgtgagatgttaggttaagtcctgcaacgagcgcaaccctcgtcgtatgttgccagcggttaggccgggcacccatacgagaccgccggcgtcaagccggaggaaggtggggacgacgtcaagtcatcatgccccttatgtcctgggctacacacgtgctacaatggccggcacaatgggctgccaacccgcgagggtgagcgaatccctaaagccggtcccagttcggattggaggctgcaacccgcctccatgaagtcggagttgctagtaatcgcggatcagcacgccgcggtgaatgcgttcccgggccttgtacacaccgcccgtcacaccacccgagtcgattgcacccgaagtcgtcggcctaaccttttaggagggagacgccgaaggtgtggttggt

SEQ ID NO: 4 - DNA, 1517 bp, Atopobium parvulum
  misc_feature: Atopobium parvulum type strain (IPP 1246)
tttgaatggagagttcgatcctggctcaggatgaacgctggcggcgcgcctaacacatgcaagtcgaacgattaaagcaccttcgggtgtgtataaagtggcgaacggctgagtaacacgtgggcaacctgcccctttcattgggatagccacgggaaaccgtggataataccgaatacttcgagacttccgcatggaagactcgagaaagctccggcggagagggatgggcccgcggcctgttagcttgttggtggggtaacggcctaccaaggcaatgatgggtagctgggttgagagaccgaccagccagattgggactgagacacggcccagactcctacgggaggcagcagtggggaatcttgcacaatgggcgaaagcctgatgcagcgacgccgcgtgcgggatgaaggccttcgggttgtaaaccgctttcagcagggacgaggcgaaagtgacggtacctgcagaagaagccccggctaactacgtgccagcagccgcggtaatacgtagggggcaagcgttatccggattcattgggcgtaaagcgctcgtaggcggtctgttaggtcgggagttaaatccgggggctcaacccccgctcgctctcgataccggcagacttgagtttggtaggggaaggtggaattcctagtgtagcggtggaatgcgcagatattaggaagaacaccagtggcgaaggcggccttctgggccataactgacgctgaggagcgaaagctaggggagcaaacaggattagataccctggtagtcctagccgtaaacgatggacactaggtgtgggggagtatttcttccgtgccgcagctaacgcattaagtgtcccgcctggggagtacggccgcaaggctaaaactcaaaggaattgacgggggcccgcacaagcagcggagcatgtggtttaattcgaagcaacgcgaagaaccttaccagggcttgacatttaggtgaagcggcggaaacgtcgtggccgaaaggagcctaaacaggtggtgcatggctgtcgtcagctcgtgtcgtgagatgttaggttaagtcctgcaacgagcgcaaccctcgtcgtatgttgccagcggttaggccgggcacccatacgagaccgccggcgtcaagccggaggaaggtggggacgacgtcaagtcatcatgccccttatgtcctgggctacacacgtgctacaatggccggcacaatgggctgccaacccgcgagggtgagcgaatccctaaagccggtcccagttcggattggaggctgcaacccgcctccatgaagtcggagttgctagtaatcgcggatcagcacgccgcggtgaatgcgttcccgggccttgtacacaccgcccgtcacaccacccgagtcgattgcacccgaagtcgtcggcctaaccttttaggagggagacgccgaaggtgtggttggtaaggggggtgaagtcgtaacaaggtagccgtaccggaaggtgcggctggatcacctcctttcta

SEQ ID NO: 5 - DNA, 1511 bp, Atopobium parvulum
  misc_feature: Atopobium parvulum type strain (IPP 1246)
atggagagttcgatcctggctcaggatgaacgctggcggcgcgcctaacacatgcaagtcgaacgattaaagcaccttcgggtgtgtataaagtggcgaacggctgagtaacacgtgggcaacctgcccctttcattgggatagccacgggaaaccgtggataataccgaatacttcgagacttccgcatggaagactcgagaaagctccggcggagagggatgggcccgcggcctgttagcttgttggtggggtaacggcctaccaaggcaatgatgggtagctgggttgagagaccgaccagccagattgggactgagacacggcccagactcctacgggaggcagcagtggggaatcttgcacaatgggcgaaagcctgatgcagcgacgccgcgtgcgggatgaaggccttcgggttgtaaaccgctttcagcagggacgaggcgaaagtgacggtacctgcagaagaagccccggctaactacgtgccagcagccgcggtaatacgtagggggcaagcgttatccggattcattgggcgtaaagcgctcgtaggcggtctgttaggtcgggagttaaatccgggggctcaacccccgctcgctctcgataccggcagacttgagtttggtaggggaaggtggaattcctagtgtagcggtggaatgcgcagatattaggaagaacaccagtggcgaaggcggccttctgggccataactgacgctgaggagcgaaagctaggggagcaaacaggattagataccctggtagtcctagccgtaaacgatggacactaggtgtgggggagtatttcttccgtgccgcagctaacgcattaagtgtcccgcctggggagtacggccgcaaggctaaaactcaaaggaattgacgggggcccgcacaagcagcggagcatgtggtttaattcgaagcaacgcgaagaaccttaccagggcttgacatttaggtgaagcggcggaaacgtcgtggccgaaaggagcctaaacaggtggtgcatggctgtcgtcagctcgtgtcgtgagatgttaggttaagtcctgcaacgagcgcaaccctcgtcgtatgttgccagcggttaggccgggcacccatacgagaccgccggcgtcaagccggaggaaggtggggacgacgtcaagtcatcatgccccttatgtcctgggctacacacgtgctacaatggccggcacaatgggctgccaacccgcgagggtgagcgaatccctaaagccggtcccagttcggattggaggctgcaacccgcctccatgaagtcggagttgctagtaatcgcggatcagcacgccgcggtgaatgcgttcccgggccttgtacacaccgcccgtcacaccacccgagtcgattgcacccgaagtcgtcggcctaaccttttaggagggagacgccgaaggtgtggttggtaaggggggtgaagtcgtaacaaggtagccgtaccggaaggtgcggctggatcacctcctttct

SEQ ID NO: 6 - DNA, 1473 bp, Atopobium parvulum
  misc_feature: Atopobium parvulum type strain (IPP 1246)
agagtttgatcctggctcaggatgaacgctggcggcgcgcctaacacatgcaagtcgaacgattaaagcaccttcgggtgtgtataaagtggcgaacggctgagtaacacgtgggcaacctgcccctttcattgggatagccacgggaaaccgtggataataccgaatacttcgagacttccgcatggaagactcgagaaagctccggcggagagggatgggcccgcggcctgttagcttgttggtggggtaacggcctaccaaggcaatgatgggtagctgggttgagagaccgaccagccagattgggactgagacacggcccagactcctacgggaggcagcagtggggaatcttgcacaatgggcgaaagcctgatgcagcgacgccgcgtgcgggatgaaggccttcgggttgtaaaccgctttcagcagggacgaggcgaaagtgacggtacctgcagaagaagccccggctaactacgtgccagcagccgcggtaatacgtagggggcaagcgttatccggattcattgggcgtaaagcgctcgtaggcggtctgttaggtcgggagttaaatccgggggctcaacccccgctcgctctcgataccggcagacttgagtttggtaggggaaggtggaattcctagtgtagcggtggaatgcgcagatattaggaagaacaccagtggcgaaggcggccttctgggccataactgacgctgaggagcgaaagctaggggagcaaacaggattagataccctggtagtcctagccgtaaacgatggacactaggtgtgggggagtatttcttccgtgccgcagctaacgcattaagtgtcccgcctggggagtacggccgcaaggctaaaactcaaaggaattgacgggggcccgcacaagcagcggagcatgtggtttaattcgaagcaacgcgaagaaccttaccagggcttgacatttaggtgaagcggcggaaacgtcgtggccgaaaggagcctaaacaggtggtgcatggctgtcgtcagctcgtgtcgtgagatgttaggttaagtcctgcaacgagcgcaaccctcgtcgtatgttgccagcggttaggccgggcacccatacgagaccgccggcgtcaagccggaggaaggtggggacgacgtcaagtcatcatgccccttatgtcctgggctacacacgtgctacaatggccggcacaatgggctgccaacccgcgagggtgagcgaatccctaaagccggtcccagttcggattggaggctgcaacccgcctccatgaagtcggagttgctagtaatcgcggatcagcacgccgcggtgaatgcgttcccgggccttgtacacaccgcccgtcacaccacccgagtcgattgcacccgaagtcgtcggcctaaccttttaggagggagacgccgaaggtgtggttggtaaggggggtgaagtcgtaacaaggtaacc

SEQ ID NO: 7 - DNA, 1531 bp, Bacteroides clarus
  misc_feature: Bacteroides clarus DSM 22519
ttacaatgaagagtttgatcctggctcaggatgaacgctagctacaggcttaacacatgcaagtcgaggggcagcggggttgaagcttgcttcaaccgccggcgaccggcgcacgggtgagtaacacgtatccaacctgccgataactccgggatagcctttcgaaagaaagattaataccggatggcatagttttcccgcatggaataactattaaagaatttcggttatcgatggggatgcgttccattaggcagttggcggggtaacggcccaccaaaccgacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggttttttgcatgtaccgtatgaataaggatcggctaactccgtgccagcagccgcggtaatacggaggatccgagcgttatccggatttattgggtttaaagggagcgtaggcggggtattaagtcagttgtgaaagtttgcggctcaaccgtaaaattgcagttgatactggtatccttgagtgcagcagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgttggcgatacaatgtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttgaattgcaactgacggaattggaaacagttctttcttcggacagttgtgaaggtgctgcatggttgtcgtcagctcgtgccgtgaggtgtcggcttaagtgccataacgagcgcaacccttatcgatagttaccatcaggtcatgctggggactctatcgagactgccgtcgtaagatgtgaggaaggtggggatgacgtcaaatcagcacggcccttacgtccggggctacacacgtgttacaatggggggtacagaaggcagctacacggcgacgtgatgctaatccccaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctttct

SEQ ID NO: 8 - DNA, 1531 bp, Bacteroides clarus; identical to SEQ ID NO: 7
  misc_feature: Bacteroides clarus DSM 22519

SEQ ID NO: 9 - DNA, 1539 bp, Bacteroides clarus
  misc_feature: Bacteroides clarus DSM 22519
cttttacaatgaagagtttgatcctggctcaggatgaacgctagctacaggcttaacacatgcaagtcgaggggcagcggggttgaagcttgcttcaaccgccggcgaccggcgcacgggtgagtaacacgtatccaacctgccgataactccgggatagcctttcgaaagaaagattaataccggatggcatagttttcccgcatggaataactattaaagaatttcggttatcgatggggatgcgttccattaggcagttggcggggtaacggcccaccaaaccgacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggttttttgcatgtaccgtatgaataaggatcggctaactccgtgccagcagccgcggtaatacggaggatccgagcgttatccggatttattgggtttaaagggagcgtaggcggggtattaagtcagttgtgaaagtttgcggctcaaccgtaaaattgcagttgatactggtatccttgagtgcagcagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgttggcgatacaatgtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttgaattgcaactgacggaattggaaacagttctttcttcggacagttgtgaaggtgctgcatggttgtcgtcagctcgtgccgtgaggtgtcggcttaagtgccataacgagcgcaacccttatcgatagttaccatcaggtcatgctggggactctatcgagactgccgtcgtaagatgtgaggaaggtggggatgacgtcaaatcagcacggcccttacgtccggggctacacacgtgttacaatggggggtacagaaggcagctacacggcgacgtgatgctaatccccaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctttctggagc

SEQ ID NO: 10 - DNA, 1479 bp, Bacteroides clarus
  misc_feature: Bacteroides clarus DSM 22519
gatgaacgctagctacaggcttaacacatgcaagtcgaggggcagcggggttgaagcttgcttcaaccgccggcgaccggcgcacgggtgagtaacacgtatccaacctgccgataactccgggatagcctttcgaaagaaagattaataccggatggcatagttttcccgcatggaataactattaaagaatttcggttatcgatggggatgcgttccattaggcagttggcggggtaacggcccaccaaaccgacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggttttttgcatgtaccgtatgaataaggatcggctaactccgtgccagcagccgcggtaatacggaggatccgagcgttatccggatttattgggtttaaagggagcgtaggcggggtattaagtcagttgtgaaagtttgcggctcaaccgtaaaattgcagttgatactggtatccttgagtgcagcagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgttggcgatacaatgtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttgaattgcaactgacggaattggaaacagttctttcttcggacagttgtgaaggtgctgcatggttgtcgtcagctcgtgccgtgaggtgtcggcttaagtgccataacgagcgcaacccttatcgatagttaccatcaggtcatgctggggactctatcgagactgccgtcgtaagatgtgaggaaggtggggatgacgtcaaatcagcacggcccttacgtccggggctacacacgtgttacaatggggggtacagaaggcagctacacggcgacgtgatgctaatccccaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaagg

SEQ ID NO: 11 - DNA, 1488 bp, Bacteroides clarus
  misc_feature: Bacteroides clarus DSM 22519
agagtttgatcctggctcaggatgaacgctagctacaggcttaacacatgcaagtcgaggggcagcggggttgaagcttgcttcaaccgccggcgaccggcgcacgggtgagtaacacgtatccaacctgccgataactccgggatagcctttcgaaagaaagattaataccggatggcatagttttcccgcatggaataactattaaagaatttcggttatcgatggggatgcgttccattaggcagttggcggggtaacggcccaccaaaccgacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggttttttgcatgtaccgtatgaataaggatcggctaactccgtgccagcagccgcggtaatacggaggatccgagcgttatccggatttattgggtttaaagggagcgtaggcggggtattaagtcagttgtgaaagtttgcggctcaaccgtaaaattgcagttgatactggtatccttgagtgcagcagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgttggcgatacaatgtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttgaattgcaactgacggaattggaaacagttctttcttcggacagttgtgaaggtgctgcatggttgtcgtcagctcgtgccgtgaggtgtcggcttaagtgccataacgagcgcaacccttatcgatagttaccatcaggtcatgctggggactctatcgagactgccgtcgtaagatgtgaggaaggtggggatgacgtcaaatcagcacggcccttacgtccggggctacacacgtgttacaatggggggtacagaaggcagctacacggcgacgtgatgctaatccccaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtaacc

SEQ ID NO: 12 - DNA, 1532 bp, Butyrivibrio crossotus
  misc_feature: Butyrivibrio crossotus T9-40 A
tttatatgagagtttgatcctggctcaggatgaacgctggcggcgtgcttaacacatgcaagtcgaacgaagcacttcataaagcttgctttaagaagtgacttagtggcggacgggtgagtaacgcgtgggtaacctgccttacacagggggataacagttagaaatgactgctaataccgcataaaacagcagagtcgcatgactcaactgtcaaagatttatcggtgtaagatggacccgcgtctgattagcttgttggtggggtaacggcctaccaaggcgacgatcagtagccggcctgagagggtgaacggccacattgggactgagacacggcccaaactcctacgggaggcagcagtggggaatattggacaatgggcgaaagcctgatccagcgacgccgcgtgagcgatgaagtatttcggtatgtaaagctctatcagcagggaagaaaacgacggtacctgactaagaagccccggctaactacgtgccagcagccgcggtaatacgtagggggcaagcgttatccggatttactgggtgtaaagggagcgtagacggcatcacaagtcagaagtgaaaatccggggctcaaccccggaactgcttttgaaactgtggagctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttgggtttcataagaagctcggtgccggcgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccgttgacctgttatgtaatgtaacatctcttcggagcaacggagacaggtggtgcatggttgtcgtcagctcgtgtcgtgagatgttgggttaagtcccgcaacgagcgcaacccttgtccttagtagccagcagtaagatgggcactctagggagactgccggggataactcggaggaaggtggggacgacgtcaaatcatcatgccccttatgacttgggctacacacgtgctacaatggcgtaaacaaagggaagcgaacctgtgaaggcaagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgtaaatgcccgaagtcggtgacctaaccgtaaggaaggagccgccgaaggcaggtatgataactggggtgaagtcgtaacaaggtagccgtatcggaaggtgcggctggatcacctcctttctaaggaa

SEQ ID NO: 13 - DNA, 1520 bp, Butyrivibrio crossotus
  misc_feature: Butyrivibrio crossotus T9-40 A
tgagagtttgatcctggctcaggatgaacgctggcggcgtgcttaacacatgcaagtcgaacgaagcacttcataaagcttgctttaagaagtgacttagtggcggacgggtgagtaacgcgtgggtaacctgccttacacagggggataacagttagaaatgactgctaataccgcataaaacagcagagtcgcatgactcaactgtcaaagatttatcggtgtaagatggacccgcgtctgattagcttgttggtggggtaacggcctaccaaggcgacgatcagtagccggcctgagagggtgaacggccacattgggactgagacacggcccaaactcctacgggaggcagcagtggggaatattggacaatgggcgaaagcctgatccagcgacgccgcgtgagcgatgaagtatttcggtatgtaaagctctatcagcagggaagaaaacgacggtacctgactaagaagccccggctaactacgtgccagcagccgcggtaatacgtagggggcaagcgttatccggatttactgggtgtaaagggagcgtagacggcatcacaagtcagaagtgaaaatccggggctcaaccccggaactgcttttgaaactgtggagctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttgggtttcataagaagctcggtgccggcgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccgttgacctgttatgtaatgtaacatctcttcggagcaacggagacaggtggtgcatggttgtcgtcagctcgtgtcgtgagatgttgggttaagtcccgcaacgagcgcaacccttgtccttagtagccagcagtaagatgggcactctagggagactgccggggataactcggaggaaggtggggacgacgtcaaatcatcatgccccttatgacttgggctacacacgtgctacaatggcgtaaacaaagggaagcgaacctgtgaaggcaagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgtaaatgcccgaagtcggtgacctaaccgtaaggaaggagccgccgaaggcaggtatgataactggggtgaagtcgtaacaaggtagccgtatcggaaggtgcggctggatcacctcctttct

SEQ ID NO: 14 - DNA, 1527 bp, Butyrivibrio crossotus
  misc_feature: Butyrivibrio crossotus T9-40 A
ctttatatgagagtttgatcctggctcaggatgaacgctggcggcgtgcttaacacatgcaagtcgaacgaagcacttcataaagcttgctttaagaagtgacttagtggcggacgggtgagtaacgcgtgggtaacctgccttacacagggggataacagttagaaatgactgctaataccgcataaaacagcagagtcgcatgactcaactgtcaaagatttatcggtgtaagatggacccgcgtctgattagcttgttggtggggtaacggcctaccaaggcgacgatcagtagccggcctgagagggtgaacggccacattgggactgagacacggcccaaactcctacgggaggcagcagtggggaatattggacaatgggcgaaagcctgatccagcgacgccgcgtgagcgatgaagtatttcggtatgtaaagctctatcagcagggaagaaaacgacggtacctgactaagaagccccggctaactacgtgccagcagccgcggtaatacgtagggggcaagcgttatccggatttactgggtgtaaagggagcgtagacggcatcacaagtcagaagtgaaaatccggggctcaaccccggaactgcttttgaaactgtggagctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttgggtttcataagaagctcggtgccggcgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccgttgacctgttatgtaatgtaacatctcttcggagcaacggagacaggtggtgcatggttgtcgtcagctcgtgtcgtgagatgttgggttaagtcccgcaacgagcgcaacccttgtccttagtagccagcagtaagatgggcactctagggagactgccggggataactcggaggaaggtggggacgacgtcaaatcatcatgccccttatgacttgggctacacacgtgctacaatggcgtaaacaaagggaagcgaacctgtgaaggcaagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgtaaatgcccgaagtcggtgacctaaccgtaaggaaggagccgccgaaggcaggtatgataactggggtgaagtcgtaacaaggtagccgtatcggaaggtgcggctggatcacctcctttct

SEQ ID NO: 15 - DNA, 1511 bp, Butyrivibrio crossotus
  misc_feature: Butyrivibrio crossotus T9-40 A
tttgatcctggctcaggatgaacgctggcggcgtgcttaacacatgcaagtcgaacgaagcacttcataaagcttgctttaagaagtgacttagtggcggacgggtgagtaacgcgtgggtaacctgccttacacagggggataacagttagaaatgactgctaataccgcataaaacagcagagtcgcatgactcaactgtcaaagatttatcggtgtaagatggacccgcgtctgattagctagttggtggggtaacggcctaccaaggcgacgatcagtagccggcctgagagggtgaacggccacattgggactgagacacggcccaaactcctacgggaggcagcagtggggaatattggacaatgggcgaaagcctgatccagcgacgccgcgtgagcgatgaagtatttcggtatgtaaagctctatcagcagggaagaaaacgacggtacctgactaagaagccccggctaactacgtgccagcagccgcggtaatacgtagggggcaagcgttatccggatttactgggtgtaaagggagcgtagacggcatcacaagtcagaagtgaaaatccggggctcaaccccggaactgcttttgaaactgtggagctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttgggtttcataagaagctcggtgccggcgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccgttgacctgttatgtaatgtaacatctcttcggagcaacggagacaggtggtgcatggttgtcgtcagctcgtgtcgtgagatgttgggttaagtcccgcaacgagcgcaacccttgtccttagtagccagcagtaagatgggcactctagggagactgccggggataactcggaggaaggtggggacgacgtcaaatcatcatgccccttatgacttgggctacacacgtgctacaatggcgtaaacaaagggaagcgaacctgtgaaggcaagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgtaaatgcccgaagtcggtgacctaaccgtaaggaaggagccgccgaaggcaggtatgataactggggtgaagtcgtaacaaggtagccgtatcggaaggtgcggctggatcacctcctt

SEQ ID NO: 16 - DNA, 1524 bp, Eubacterium hadrum
  misc_feature: Eubacterium hadrum B2-52
tttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtaggggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctttct

SEQ ID NO: 17 - DNA, 1524 bp, Eubacterium hadrum
  misc_feature: Eubacterium hadrum B2-52
tttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctaccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccttatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggccttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctttct

SEQ ID NO: 18 - DNA, 1514 bp, Eubacterium hadrum
  misc_feature: Eubacterium hadrum B2-52
tggctcaggatgaacgctggcggcgtgcttaacacatgcaagtcgaacgaagctgcttaactgatcttcttcggaattgacgttttgtagactgagtggcggacgggtgagtaacgcgtgggcaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatttcaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctccttt

SEQ ID NO: 19 - DNA, 1488 bp, Prevotella stercorea
  misc_feature: Prevotella stercorea CB35
agagtttgatcctggctcaggatgaacgctagctacaggcttaacacatgcaagtcgaggggcagcatgtcggttgcttgcaaccgatgatggcgaccggcgcacgggtgagtaacgcgtatccaacctacccttgtccatcggataacccgtcgaaaggcggcctaacacgatatgcggttcacagcaggcatctaacgtgaacgaaatgtgaaggagaaggatggggatgcgtctgattagcttgttggcggggtaacggcccaccaaggctacgatcagtaggggttctgagaggaaggtcccccacattggaactgagacacggtccaaactcctacgggaggcagcagtgaggaatattggtcaatggacgagagtctgaaccagccaagtagcgtgcaggatgacggccctatgggttgtaaactgcttttatagggggataaagtgtgccacgtgtggcatattgcaggtaccctatgaataaggaccggctaattccgtgccagcagccgcggtaatacggaaggtccgggcgttatccggatttattgggtttaaagggagcgtaggccgtttggtaagcgtgttgtgaaatgtcggggctcaacctgggcattgcagcgcgaactgccagacttgagtgcgcaggaagtaggcggaattcgtcgtgtagcggtgaaatgcttagatatgacgaagaactccgattgcgaaggcagcctgctgtagcgcaactgacgctgaagctcgaaagcgtgggtatcgaacaggattagataccctggtagtccacgcggtaaacgatggatgctcgctgtttgcgtctgacgtaagcggccaagcgaaagcgttaagcatcccacctggggagtacgccggcaacggtgaaactcaaaggaattgacgggggcccgcacaagcggaggaacatgtggtttaattcgatgatacgcgaggaaccttacccgggcttgaactgtaggcgaacgattcagagatgatgaggcccttcggggcgcctatggaggtgctgcatggttgtcgtcagctcgtgccgtgaggtgtcggcttaagtgccataacgagcgcaacccttgttcgtagttgccatcgggtgatgccgggcactctgcgaagactgcctccgtaaggagtgaggaaggtggggatgacgtcaaatcagcacggcccttacgtccggggctacacacgtgttacaatggcatgtacagaaagcggggtgtatgcaaatacgctcgaatccctaaaacatgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgcgagggtcggcctagggtgaaaccggtgattggggctaagtcgtaacaaggtaacc

SEQ ID NO: 20 - DNA, 1444 bp, Roseburia inulinivorans
  misc_feature: Roseburia inulinivorans A2-194
ttgatcctggctcaggatgaacgctggcggcgtgcttaacacatgcaagtcgaacgaagcacttttacagatttcttcggaatgaagttttagtgactgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggcagggcaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggaaagcacagcttttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggacagtaatgtgtcctttccttcgggacagaagtgacaggtggtgcatggttgtcgtcagctcgtgtcgtgagatgttgggttaagtcccgcaacgagcgcaacccttatccccagtagccagcggttcggacgggcactctgaggagactgccagggataacctggaggaaggtggggatgacgtcaaatcatcatgccccttatgacttgggctacacacgtgctacaatggcgtaaacaaagggaagcgagaccgtgaggtggagcaaatcccaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgc

SEQ ID NO: 21 - DNA, 1531 bp, Ruminococcus
  misc_feature: Ruminococcus sp. 5.1.39BFAA
ttttatcagagagtttgatcctggctcaggatgaacgctggcggcgtgcttaacacatgcaagtcgaacgggaattactttattgacttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcacctcctttct

SEQ ID NO: 22 - DNA, 1540 bp, Ruminococcus
  misc_feature: Ruminococcus sp. 5.1.39BFAA
ttttatcagagagtttgatcctggctcaggatgaacgctggcggcgtgcttaacacatgcaagtcgaacgggaaatatttcattgagacttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctggttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccggaacttaatcgttcctttccttcgggacagaggagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcacctcctttctaaggaa

SEQ ID NO: 23 - DNA, 1330 bp, Ruminococcus
  misc_feature: Ruminococcus sp. 5.1.39BFAA
ttttatcagagagtttgatcctggctcaggatgaacgctggcggcgtgcttaacacatgcaagtcgaacgggaaatatttcattgagacttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctggttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaa

SEQ ID NO: 24 - DNA, 1540 bp, Ruminococcus
  misc_feature: Ruminococcus sp. 5.1.39BFAA
tttaatcagagagtttgatcctggctcaggatgaacgctggcggcgtgcttaacacatgcaagtcgaacgggaattactttattgaaacttcggtcgatttaatttaattctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctagttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcacctcctttctaaggaa

SEQ ID NO: 25 - DNA, 1540 bp, Ruminococcus
  misc_feature: Ruminococcus sp. 5.1.39BFAA
ttttatcagagagtttgatcctggctcaggatgaacgctggcggcgtgcttaacacatgcaagtcgaacgggaattactttattgaaacttcggtcgatttaatttaattctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtgaggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccggaacttaatcgttcctttccttcgggacagaggagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcacctcctttctaaggaa

SEQ ID NO: 26 - DNA, 1541 bp, Ruminococcus
  misc_feature: Ruminococcus sp. 5.1.39BFAA
ttttatcagagagtttgatcctggctcaggatgaacgctggcggcgtgcttaacacatgcaagtcgaacgggaattactttattgaaacttcggtcgatttaatttaattctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctagttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgactgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatggggagtcagtaacgcccgaagtcagtgacctaaccgaaagggaggagctgccgaaggcgggaccgatgactggggtgaagtcgtaacaaggtagccgtatcggaaggtgcggctggatcacctcctttctaaggaa

SEQ ID NO: 27 - DNA, 1540 bp, Ruminococcus; identical to SEQ ID NO: 24
  misc_feature: Ruminococcus sp. 5.1.39BFAA

SEQ ID NO: 28 - DNA, 1540 bp, Ruminococcus
  misc_feature: Ruminococcus sp. 5.1.39BFAA
tttaatcagagagtttgatcctggctcaggatgaacgctggcggcgtgcttaacacatgcaagtcgaacgggaattactttattgaaacttcggtcgatttaatttaattctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctggttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtagcatagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgaaacttaatcggatctttccttcgggacagacgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcacctcctttctaaggaa

SEQ ID NO: 29 - DNA, 1523 bp, Ruminococcus
  misc_feature: Ruminococcus sp. 5.1.39BFAA
gagagtttgatcctggctcaggatgaacgctggcggcgtgcttaacacatgcaagtcgaacgggaattactttattgacttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcacctcctttct

SEQ ID NO: 30 - DNA, 1527 bp, Zinderia insecticola
  misc_feature: Zinderia insecticola CARI
atataaataagagtttgatcctggctcagattgaacgctagcggtatgctttacacatgcaagtcgaacgacaatattaaagcttgctttaatataaagtggcgaacgggtgagtaatatatcaaaacgtaccttaaagtgggggataactaattgaaaaattagataataccgcatattaatcttaggatgaaaataggaataatatcttatgcttttagatcggttgatatctgattagctagttggtagggtaaatgcttaccaaggcaatgatcagtagctggttttagcgaatgatcagccacactggaactgagacacggtccagacttctacggaaggcagcagtggggaatattggacaatgggagaaatcctgatccagcaataccgcgtgagtgatgaaggccttagggtcgtaaaactcttttgttaggaaagaaataattttaaataatatttaaaattgatgacggtacctaaagaataagcaccggctaactacgtgccagcagccgcggtaatacgtagggtgcaagcgttaatcggaattattgggcgtaaagagtgcgtaggctgttatataagatagatgtgaaatacttaagcttaacttaagaactgcatttattactgtttaactagagtttattagagagaagtggaattttatgtgtagcagtgaaatgcgtagatatataaaggaatatcgatggcgaaggcagcttcttggaataatactgacgctgaggcacgaaagcgtggggagcaaacaggattagataccctggtagtccacgccctaaactatgtctactagttattaaattaaaaataaaatttagtaacgtagctaacgcattaagtagaccgcctggggagtacgatcgcaagattaaaactcaaaggaattgacggggacccgcacaagcggtggatgatgtggattaattcgatgcaacacgaaaaaccttacctactcttgacatgtttggaattttaaagaaatttaaaagtgcttgaaaaagaaccaaaacacaggtgctgcatggctgtcgtcagctcgtgtcgtgagatgttgggttaagtcccgcaacgagcgcaacccttgttattatttgctaataaaaagaactttaataagactgccaatgacaaattggaggaaggtggggatgacgtcaagtcctcatggcccttatgagtagggcttcacacgtcatacaatgatatatacaatgggtagcaaatttgtgaaaatgagccaatccttaaagtatatcttagttcggattgtagtctgcaactcgactacatgaagttggaatcgctagtaatcgcggatcagcatgccgcggtgaatacgttctcgggtcttgtacacaccgcccgtcacaccatggaagtgatttttaccagaaattatttgtttaacctttattggaaaaaaataattaaggtagaattcatgactggggtgaagtcgtaacaaggtagcagtatcggaaggtgcggctggattac

SEQ ID NO: 31 - DNA, 1520 bp, Zinderia insecticola
  misc_feature: Zinderia insecticola CARI
taagagtttgatcctggctcagattgaacgctagcggtatgctttacacatgcaagtcgaacgacaatattaaagcttgctttaatataaagtggcgaacgggtgagtaatatatcaaaacgtaccttaaagtgggggataactaattgaaaaattagataataccgcatattaatcttaggatgaaaataggaataatatcttatgcttttagatcggttgatatctgattagctagttggtagggtaaatgcttaccaaggcaatgatcagtagctggttttagcgaatgatcagccacactggaactgagacacggtccagacttctacggaaggcagcagtggggaatattggacaatgggagaaatcctgatccagcaataccgcgtgagtgatgaaggccttagggtcgtaaaactcttttgttaggaaagaaataattttaaataatatttaaaattgatgacggtacctaaagaataagcaccggctaactacgtgccagcagccgcggtaatacgtagggtgcaagcgttaatcggaattattgggcgtaaagagtgcgtaggctgttatataagatagatgtgaaatacttaagcttaacttaagaactgcatttattactgtttaactagagtttattagagagaagtggaattttatgtgtagcagtgaaatgcgtagatatataaaggaatatcgatggcgaaggcagcttcttggaataatactgacgctgaggcacgaaagcgtggggagcaaacaggattagataccctggtagtccacgccctaaactatgtctactagttattaaattaaaaataaaatttagtaacgtagctaacgcattaagtagaccgcctggggagtacgatcgcaagattaaaactcaaaggaattgacggggacccgcacaagcggtggatgatgtggattaattcgatgcaacacgaaaaaccttacctactcttgacatgtttggaattttaaagaaatttaaaagtgcttgaaaaagaaccaaaacacaggtgctgcatggctgtcgtcagctcgtgtcgtgagatgttgggttaagtcccgcaacgagcgcaacccttgttattatttgctaataaaaagaactttaataagactgccaatgacaaattggaggaaggtggggatgacgtcaagtcctcatggcccttatgagtagggcttcacacgtcatacaatgatatatacaatgggtagcaaatttgtgaaaatgagccaatccttaaagtatatcttagttcggattgtagtctgcaactcgactacatgaagttggaatcgctagtaatcgcggatcagcatgccgcggtgaatacgttctcgggtcttgtacacaccgcccgtcacaccatggaagtgatttttaccagaaattatttgtttaacctttattggaaaaaaataattaaggtagaattcatgactggggtgaagtcgtaacaaggtagcagtatcggaaggtgcggctggattac

SEQ ID NO: 32 - DNA, 1194 bp, Coprobacillus
  misc_feature: Coprobacillus sp. 29_1
gaggcagcagtagggaattttcggcaatgggggaaaccctgaccgagcaacgccgcgtgagggaagaagtatttcggtatgtaaacctctgttataaaggaagaacggtatgaataggaaatgattcataagtgacggtactttatgagaaagccacggctaactacgtgccagcagccgcggtaatacgtaggtggcgagcgttatccggaatcattgggcgtaaagagggagcaggcggcaatagaggtctgcggtgaaagcctgaagctaaacttcagtaagccgtggaaaccaaatagctagagtgcagtagaggatcgtggaattccatgtgtagcggtgaaatgcgtagatatatggaggaacaccagtggcgaaggcgacgatctgggctgcaactgacgctcagtcccgaaagcgtggggagcaaataggattagataccctagtagtccacgccgtaaacgatgagtactaagtgttgggggtcaaacctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatacctctaaaggctctagagatagagagatagctataggggatacaggtggtgcatggttgtcgtcagctcgtgtcgtgagatgttgggttaagtcccgcaacgagcgcaacccttgtcgctagttaccatcattaagttggggactctagcgagactgcctctgcaaggaggaggaaggcggggatgacgtcaaatcatcatgccccttatgacctgggctacacacgtgctacaatggacggatcaaagggaagcgaagccgcgaggtggagcgaaacccaaaaacccgttctcagttcggactgcagtctgcaactcgactgcacgaagttggaatcgctagtaatcgcgaatcagaatgtcgcggtgaatacgttctcgggccttgtacacaccgcccgtcacaccatgagagttggtaacacccgaagccggtggcttaaccgcaaggagagagctgtctaaggtgggactgatgattagggtgaagtcgtaacaaggtatccctacgggaacgtggggatggatcacctcctttctagggaga

SEQ ID NO: 33 - DNA, 1334 bp, Dorea longicatena
  misc_feature: Dorea longicatena 111-35
gagcggcggacgggtgagtaacgcgtgggtaacctgcctcatacagggggataacagttagaaatgactgctaataccgcataagaccacgtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcataacgcccgaagtcagtgacccaaccgtaagg

SEQ ID NO: 34 - DNA, 1540 bp, Dorea longicatena
  misc_feature: Dorea longicatena 111-35
tttaacgagagtttgatcctggctcaggatgaacgctggcggcgtgcttaacacatgcaagtcgagcgaagcgcttaagtttgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctcctttctaaggaa

SEQ ID NO: 35 - DNA, 1528 bp, Dorea longicatena
  misc_feature: Dorea longicatena 111-35
gagagtttgatcctggctcaggatgaacgctggcggcgtgcttaacacatgcaagtcgagcgaagcgcttaagtttgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctcctttct

SEQ ID NO: 36 - DNA, 1528 bp, Dorea longicatena; identical to SEQ ID NO: 35
  misc_feature: Dorea longicatena 111-35

SEQ ID NO: 37 - DNA, 1528 bp, Eubacterium limosum
  misc_feature: Eubacterium limosum KIST612
tttttattgagagtttgatcctggctcaggacgaacgctggcggtatgcttaacacatgcaagtcgaacgagaaggttttgaatgatccttcgggtgaaattagaactggaaagtggcgaacgggtgagtaacgcgtgggtaacctgccctatggaaaggaatagcctcgggaaactgggagtaaagccttatattatggttttgtcgcatggcgagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaacatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggatgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtagccgtatcggaaggtgcggctggatcacctcctttct

SEQ ID NO: 38 - DNA, 1528 bp, Eubacterium limosum
  misc_feature: Eubacterium limosum KIST612
ttttattgagagtttgatcctggctcaggacgaacgctggcggtatgcttaacacatgcaagtcgaacgagaaggttttgaatgatccttcgggtgaaattagaactggaaagtggcgaacgggtgagtaacgcgtgggtaacctgccctatggaaaggaatagcctcgggaaactgggagtaaagccttatattatggttttgtcgcatggcgagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaacatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggatgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtagccgtatcggaaggtgcggctggatcacctcctttcta

SEQ ID NO: 39 - DNA, 1528 bp, Eubacterium limosum; identical to SEQ ID NO: 38
  misc_feature: Eubacterium limosum KIST612

SEQ ID NO: 40 - DNA, 1528 bp, Eubacterium limosum; identical to SEQ ID NO: 38
  misc_feature: Eubacterium limosum KIST612

SEQ ID NO: 41 - DNA, 1528 bp, Eubacterium limosum
  misc_feature: Eubacterium limosum KIST612
ttttattgagagtttgatcctggctcaggacgaacgctggcggtatgcttaacacatgcaagtcgaacgagaaagttttgaatgatccttcgggtgaaattagaactggaaagtggcgaacgggtgagtaacgcgtgggtaacctgccctatggaaaggaatagcctcgggaaactgggagtaaagccttatattatggttttgtcgcatggcgagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaacatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggatgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtagccgtatcggaaggtgcggctggatcacctcctttcta

SEQ ID NO: 42 - DNA, 1528 bp, Eubacterium limosum
  misc_feature: Eubacterium limosum KIST612
atttattgagagtttgatcctggctcaggacgaacgctggcggtatgcttaacacatgcaagtcgaacgagaaagttttgaatgatccttcgggtgaaattagaactggaaagtggcgaacgggtgagtaacgcgtgggtaacctgccctatggaaaggaatagcctcgggaaactgggagtaaagccttatattatggttttgtcgcatggcgagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaacatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggatgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtagccgtatcggaaggtgcggctggatcacctcctttcta

SEQ ID NO: 43 - DNA, 1528 bp, Eubacterium limosum; identical to SEQ ID NO: 37
  misc_feature: Eubacterium limosum KIST612

SEQ ID NO: 44 - DNA, 1542 bp, Hungatella hathewayi
  misc_feature: Hungatella hathewayi 12489931
tttaacatgagagttcgatcctggctcaggatgaacgctggcggcgtgcttaacacatgcaagtcgagcgaagcggtttcaatgaagttttcggatggaattgaaattgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaacgtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaacactttaaccggtgtccctcttcggagcaatggagacaggtggtgcatggttgtcgtcagctcgtgtcgtgagatgttgggttaagtcccgcaacgagcgcaacccttatccttagtagccagcacatgatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggagggagctgccgaaggcgggactgataactggggtgaagtcgtaacaaggtagccgtatcggaaggtgcggctggatcacctcctttctaaggaa

SEQ ID NO: 45 - DNA, 1537 bp, Lachnospiraceae bacterium
  misc_feature: Lachnospiraceae bacterium 5/1/63FAA
ttgaacatgagagtttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctttct

SEQ ID NO: 46 - DNA, 1541 bp, Lachnospiraceae bacterium
  misc_feature: Lachnospiraceae bacterium 5/1/63FAA
gaacatgagagtttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctttctaaggaa

SEQ ID NO: 47 - DNA, 1537 bp, Lachnospiraceae bacterium; identical to SEQ ID NO: 45
  misc_feature: Lachnospiraceae bacterium 5/1/63FAA

(19) United States
(12) Patent Application Publication
(10) Pub. No.: US 2023/0035667 A1
(43) Pub. Date: Feb. 2, 2023

(54) METHODS AND COMPOSITIONS FOR TREATING TYPE 2 DIABETES

(71) Applicant: Second Genome, Inc., Brisbane, CA (US)

(72) Inventors: Jayamary Divya Ravichandar, Brisbane, CA (US); Shoko Kawana, San Mateo, CA (US); Mitsuko Lynn Yamamoto, Brisbane, CA (US)

(21) Appl. No.: 17/758,748

(22) PCT Filed: Jan. 15, 2021
(86) PCT No.: PCT/US21/13741

§ 371 Date: Jul. 13, 2022

Related U.S. Application Data
(60) Provisional application No. 62/962,805, filed on Jan. 17, 2020.

(51) Int. Cl. A61K 35/741; G01N 33/569; A61P 3/10; C12N 7/00

(52) U.S. Cl. CPC A61K 35/741; G01N 33/56911; A61P 3/10; C12N 7/00; G01N 2333/195; G01N 2800/042

(57) ABSTRACT

Methods and compositions are provided herein for treating type 2 diabetes in a subject, using one or more bacterial strains such as Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARI.

### CROSS-REFERENCE TO RELATED APPLICATIONS

This application claims priority to U.S. Provisional Patent Application Ser. No. 62/962,805, filed Jan. 17, 2020; the entire contents of which are herein incorporated by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: 47192_0047WO1_SequenceListing.txt, date created, Jan. 15, 2021, file size ≈100 kilobytes.

### TECHNICAL FIELD

The present disclosure is related to bacterial strains and compositions thereof, and using such bacterial strains and compositions thereof for treating type 2 diabetes in a subject.

### BACKGROUND

The human microbiome comprises a diverse array of microorganisms, primarily prokaryotes, which play a significant role in the health of the host organism. The complexity of the microbiome, in terms of both its population makeup and composite function, has recently become an intense area of study as research increasingly shows that manipulation of the microbiome can provide health benefits and may be effective in treating a number of diseases and disorders. Currently, a number of probiotics are marketed which contain live bacteria and yeast and are believed to augment the benefits of these microbes which naturally occur in the human body. Increasingly, live biotherapeutic products (LBPs) are being developed for controlled clinical studies and regulatory approval in the treatment of disease.

Gut microbiome dysbiosis can be a factor in the rapid progression of insulin resistance in type 2 diabetes (Sharma and Tripathi. J Nutr Biochem. 2019 January; 63:101-108). As of 2017 approximately 30.3 million people have diabetes. Improved therapies such as microbe-based therapies are desirable.

### SUMMARY

Provided herein are methods and compositions for treating a subject in need thereof.

Also provided herein are methods of identifying a subject as having or having an increased likelihood of developing type 2 diabetes that include (a) identifying a subject having a sample that has (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, as having or having an increased likelihood of developing type 2 diabetes; or (b) identifying a subject having a sample that does not have (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as not having or not having an increased likelihood of developing type 2 diabetes.

Also provided herein are methods of diagnosing a subject as having type 2 diabetes that include (a) identifying a subject having a sample that has (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as having type 2 diabetes; or (b) identifying a subject having a sample that does not have (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as not having type 2 diabetes.

Also provided herein are methods of treating type 2 diabetes in a subject that include (a) administering a type 2 diabetes therapy to a subject determined to have a sample that has (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or (b) not administering a type 2 diabetes therapy to a subject determined not to have a sample that has (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

Also provided herein are methods for treating a subject in need thereof that include (a) administering a composition comprising an effective amount of a bacterial species selected from the group consisting of Alistipes sp., Atopobium parvulum type, Bacteroides clarus, Butyrivibrio crossotus, Eubacterium hadrum, Prevotella stercorea, Roseburia inulinivorans, Ruminococcus sp., and Zinderia insecticola, as a monotherapy, or in conjunction with another type 2 diabetes therapy, to a subject determined to have a sample that has (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or (b) not administering a composition comprising an effective amount of a bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as a monotherapy, or in conjunction with another type 2 diabetes therapy, to a subject determined not to have a sample that has (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

In some embodiments of any of the methods herein, the subject has type 2 diabetes.

In some embodiments of any of the methods herein, the method comprises detecting the level of one or more bacterial species in the sample from the subject. In some embodiments of any of the methods herein, the level of Alistipes sp., Atopobium parvulum, Bacteroides clarus, Butyrivibrio crossotus, Eubacterium hadrum, Prevotella stercorea, Roseburia inulinivorans, Ruminococcus sp., or Zinderia insecticola is increased in comparison to the same bacterial species in a reference sample.

In some embodiments of any of the methods herein, the method comprises determining that the sample has (i) an increased level of two or more (e.g., three or more, four or more, or five) of: Coprobacillus, Dorea longicatena, Eubacterium limosum, Hungatella hathewayi, and Lachnospiraceae bacterium; and/or (ii) a decreased level of two or more (e.g., three or more, four or more, five or more, six or more, seven or more, eight or more, or nine) of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

In some embodiments of any of the methods herein, the method comprises determining that the sample has (i) an increased level of three or more (e.g. four or more, or five) of: Coprobacillus, Dorea longicatena, Eubacterium limosum, Hungatella hathewayi, and Lachnospiraceae bacterium; and/or (ii) a decreased level of three or more (four or more, five or more, six or more, seven or more, eight or more, or nine) of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

In some embodiments of any of the methods herein, the method comprises determining that the sample has (i) an increased level of four or more (e.g. five) of: Coprobacillus, Dorea longicatena, Eubacterium limosum, Hungatella hathewayi, and Lachnospiraceae bacterium; and/or (ii) a decreased level of four or more (e.g. five or more, six or more, seven or more, eight or more, or nine) of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

Also provided herein are methods for treating type 2 diabetes in a subject that include administering to the subject a composition comprising an effective amount of a bacterial strain selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, and a combination thereof.

Also provided herein are methods for treating type 2 diabetes in a subject that include (a) detecting a dysbiosis associated with type 2 diabetes in a sample from the subject; and (b) administering to the subject a composition comprising an effective amount of a bacterial strain selected from the group consisting of Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, and a combination thereof.

In some embodiments of any of the methods herein, the sample is a fecal sample.

In some embodiments of any of the methods herein, detecting the dysbiosis associated with type 2 diabetes comprises determining bacterial gene expression in the sample from the subject. In some embodiments of any of the methods herein, detecting the dysbiosis associated with type 2 diabetes comprises determining bacterial composition in the sample from the subject. In some embodiments of any of the methods herein, detecting the dysbiosis associated with type 2 diabetes comprises determining that Coprobacillus sp., Dorea longicatena, Eubacterium limosum, Hungatella hathewayi, Lachnospiraceae bacterium, or a combination thereof, is increased in the sample from subject. In some embodiments of any of the methods herein, detecting the dysbiosis associated with type 2 diabetes comprises determining that Alistipes sp., Atopobium parvulum type strain, Bacteroides clarus, Butyrivibrio crossotus, Eubacterium hadrum, Prevotella stercorea, Roseburia inulinivorans, Ruminococcus sp., Zinderia insecticola, or a combination thereof, is decreased in the sample from subject.

In some embodiments of any of the methods herein, Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, or a combination thereof, is decreased in the gastrointestinal tract of the subject.

Also provided herein are methods for treating a subject in need thereof that include decreasing a population of an increased bacterial species in the subject, wherein the increased bacterial species is selected from the group consisting of Coprobacillus sp., Dorea longicatena, Eubacterium limosum, Hungatella hathewayi, Lachnospiraceae bacterium, and a combination thereof.

In some embodiments of any of the methods herein, the subject has type 2 diabetes.

In some embodiments of any of the methods herein, decreasing the population of an increased bacterial strain comprises administering to the subject a bacteriophage. In some embodiments of any of the methods herein, decreasing the population of an increased bacterial species comprises administering to the subject a composition comprising an effective amount of a bacterial species selected from the group consisting of Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, and a combination thereof.

In some embodiments of any of the methods herein, the bacterial species Alistipes sp. comprises the bacterial strain Alistipes sp. HGB5. In some embodiments of any of the methods herein, the bacterial species Atopobium parvalum comprises the bacterial strain Atopobium parvulum type strain (IPP 1246). In some embodiments of any of the methods herein, the bacterial species Bacteroides clarus comprises the bacterial strain Bacteroides clarus DSM 22519. In some embodiments of any of the methods herein, the bacterial species Butyrivibrio crossotus comprises the bacterial strain Butyrivibrio crossotus T9-40 A. In some embodiments of any of the methods herein, the bacterial species Eubacterium hadrum comprises the bacterial strain Eubacterium hadrum B2-52. In some embodiments of any of the methods herein, the bacterial species Prevotella stercorea comprises the bacterial strain Prevotella stercorea CB35. In some embodiments of any of the methods herein, the bacterial species Roseburia inulinivorans comprises the bacterial strain Roseburia inulinivorans A2-194. In some embodiments of any of the methods herein, the bacterial species Ruminococcus sp. comprises the bacterial strain Ruminococcus sp. 5.1.39BFAA. In some embodiments of any of the methods herein, the bacterial species Zinderia insecticola comprises the bacterial strain Zinderia insecticola CARI.

In some embodiments of any of the methods herein, the bacterial strain improves intestinal barrier function of the subject.

In some embodiments of any of the methods herein, the Alistipes sp. HGB5 has a 16S RNA gene that is at least 95% identical to SEQ ID NO:1. In some embodiments of any of the methods herein, the Alistipes sp. HGB5 has a 16S RNA gene that is at least 95% identical to SEQ ID NO:2.

In some embodiments of any of the methods herein, the Atopobium parvulum type strain (IPP 1246) has a 16S RNA gene that is at least 95% identical to SEQ ID NO:3. In some embodiments of any of the methods herein, the Atopobium parvulum type strain (IPP 1246) has a 16S RNA gene that is at least 95% identical to SEQ ID NO:4. In some embodiments of any of the methods herein, the Atopobium parvulum type strain (IPP 1246) has a 16S RNA gene that is at least 95% identical to SEQ ID NO:5. In some embodiments of any of the methods herein, the Atopobium parvulum type strain (IPP 1246) has a 16S RNA gene that is at least 95% identical to SEQ ID NO:6.

In some embodiments of any of the methods herein, the Bacteroides clarus DSM 22519 has a 16S RNA gene that is at least 95% identical to SEQ ID NO:7. In some embodiments of any of the methods herein, the Bacteroides clarus DSM 22519 has a 16S RNA gene that is at least 95% identical to SEQ ID NO:8. In some embodiments of any of the methods herein, the Bacteroides clarus DSM 22519 has a 16S RNA gene that is at least 95% identical to SEQ ID NO:9. In some embodiments of any of the methods herein, the Bacteroides clarus DSM 22519 has a 16S RNA gene that is at least 95% identical to SEQ ID NO:10. In some embodiments of any of the methods herein, the Bacteroides clarus DSM 22519 has a 16S RNA gene that is at least 95% identical to SEQ ID NO:11.

In some embodiments of any of the methods herein, the Butyrivibrio crossotus T9-40 A has a 16S RNA gene that is at least 95% identical to SEQ ID NO:12. In some embodiments of any of the methods herein, the Butyrivibrio crossotus T9-40 A has a 16S RNA gene that is at least 95% identical to SEQ ID NO:13. In some embodiments of any of the methods herein, the Butyrivibrio crossotus T9-40 A has a 16S RNA gene that is at least 95% identical to SEQ ID NO:14. In some embodiments of any of the methods herein, the Butyrivibrio crossotus T9-40 A has a 16S RNA gene that is at least 95% identical to SEQ ID NO:15.

In some embodiments of any of the methods herein, the Eubacterium hadrum B2-52 has a 16S RNA gene that is at least 95% identical to SEQ ID NO:16. In some embodiments of any of the methods herein, the Eubacterium hadrum B2-52 has a 16S RNA gene that is at least 95% identical to SEQ ID NO:17. In some embodiments of any of the methods herein, the Eubacterium hadrum B2-52 has a 16S RNA gene that is at least 95% identical to SEQ ID NO:18.

In some embodiments of any of the methods herein, the Prevotella stercorea CB35 has a 16S RNA gene that is at least 95% identical to SEQ ID NO:19.

In some embodiments of any of the methods herein, the Roseburia inulinivorans A2-194 has a 16S RNA gene that is at least 95% identical to SEQ ID NO:20.

In some embodiments of any of the methods herein, the Ruminococcus sp. 5.1.39BFAA has a 16S RNA gene that is at least 95% identical to SEQ ID NO:21. In some embodiments of any of the methods herein, the Ruminococcus sp. 5.1.39BFAA has a 16S RNA gene that is at least 95% identical to SEQ ID NO:22. In some embodiments of any of the methods herein, the Ruminococcus sp. 5.1.39BFAA has a 16S RNA gene that is at least 95% identical to SEQ ID NO:23.

In some embodiments of any of the methods herein, the Ruminococcus sp. 5.1.39BFAA has a 16S RNA gene that is at least 95% identical to SEQ ID NO:24. In some embodiments of any of the methods herein, the Ruminococcus sp. 5.1.39BFAA has a 16S RNA gene that is at least 95% identical to SEQ ID NO:25.

In some embodiments of any of the methods herein, the Ruminococcus sp. 5.1.39BFAA has a 16S RNA gene that is at least 95% identical to SEQ ID NO:26. In some embodiments of any of the methods herein, the Ruminococcus sp. 5.1.39BFAA has a 16S RNA gene that is at least 95% identical to SEQ ID NO:27. In some embodiments of any of the methods herein, the Ruminococcus sp. 5.1.39BFAA has a 16S RNA gene that is at least 95% identical to SEQ ID NO:28. In some embodiments of any of the methods herein, the Ruminococcus sp. 5.1.39BFAA has a 16S RNA gene that is at least 95% identical to SEQ ID NO:29.

In some embodiments of any of the methods herein, the Zinderia insecticola CARI has a 16S RNA gene that is at least 95% identical to SEQ ID NO:30. In some embodiments of any of the methods herein, the Zinderia insecticola CARI has a 16S RNA gene that is at least 95% identical to SEQ ID NO:31.

In some embodiments of any of the methods herein, the bacterial species in the composition is viable. In some embodiments of any of the methods herein, the bacterial species is lyophilized.

In some embodiments of any of the methods herein, the composition further comprises one or more cryopreservants.

In some embodiments of any of the methods herein, the therapeutically effective amount of the bacterial species comprises at least about 1×10³ colony forming units (CFU) of the bacterial species. In some embodiments of any of the methods herein, the therapeutically effective amount of the bacterial species comprises about 1×10⁴ to about 1×10¹⁵ CFU of the bacterial species. In some embodiments of any of the methods herein, the therapeutically effective amount of the bacterial species comprises about 1×10⁶ to about 1×10¹⁰ CFU of the bacterial species.

In some embodiments of any of the methods herein, the bacterial species in the composition is non-viable. In some embodiments of any of the methods herein, the non-viable bacterial species is heat-killed, irradiated, or lysed.

In some embodiments of any of the methods herein, the method comprises administering the composition to the subject once, twice, or three times per day. In some embodiments of any of the methods herein, the composition is formulated for oral administration. In some embodiments of any of the methods herein, the composition is formulated as a tablet, a capsule, a powder, or a liquid. In some embodiments of any of the methods herein, the composition is formulated as a tablet. In some embodiments of any of the methods herein, the tablet is coated. In some embodiments of any of the methods herein, the coating comprises an enteric coating.

In some embodiments of any of the methods herein, the method further comprises administering another treatment of type 2 diabetes and/or other adjunct therapy to the subject. In some embodiments of any of the methods herein, the composition comprising the bacterial species treatment and the treatment for type 2 diabetes and/or adjunct therapy are administered simultaneously. In some embodiments of any of the methods herein, the composition comprising the bacterial species treatment and the treatment for type 2 diabetes and/or adjunct therapy are administered sequentially.

In some embodiments of any of the methods herein, the treatment for type 2 diabetes and/or adjunct therapy comprises a probiotic.

In some embodiments of any of the methods herein, the treatment for type 2 diabetes and/or adjunct therapy comprises a therapeutic agent. In some embodiments of any of the methods herein, the therapeutic agent comprises metformin, a sulfonylurea, a meglitinide, a thiazolidinedione, a DPP-4 inhibitor, a GLP-1 receptor agonist, a SGLT-2 inhibitor, insulin, or a combination thereof.

In some embodiments of any of the methods herein, the therapeutic agent comprises metformin, glyburide, glipizide, glimepiride, repaglinide, nateglinide, rosiglitazone, pioglitazone, sitagliptin, saxagliptin, linagliptin, exenatide, liraglutide, semaglutide, canagliflozin, dapagliflozin, empagliflozin, insulin, or a combination thereof.

In some embodiments of any of the methods herein, the composition comprising the bacterial species further comprises the therapeutic agent.

In some embodiments of any of the methods herein, the subject is a human.

Also provided herein are methods for treating a subject in need thereof that include administering to the subject a composition comprising an effective amount of a bacterial strain selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof. In some embodiments of any of the methods herein, the subject has type 2 diabetes.

Also provided herein are methods for treating type 2 diabetes in a subject that include administering to the subject a composition comprising an effective amount of a bacterial strain selected from the group consisting of: Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

Also provided herein are methods for treating type 2 diabetes in a subject that include (a) detecting a dysbiosis associated with type 2 diabetes in a sample from the subject; and (b) administering to the subject a composition comprising an effective amount of a bacterial strain selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

In some embodiments of any of the methods herein, the sample is a fecal sample.

In some embodiments of any of the methods herein, detecting the dysbiosis associated with type 2 diabetes comprises determining bacterial gene expression in the sample from the subject. In some embodiments of any of the methods herein, detecting the dysbiosis associated with type 2 diabetes comprises determining bacterial composition in the sample from the subject. In some embodiments of any of the methods herein, detecting the dysbiosis associated with type 2 diabetes comprises determining that Coprobacillus sp. 291, Dorea longicatena 111-35, Eubacterium limosum KIST612, Hungatella hathewayi 12489931, Lachnospiraceae bacterium 5/1/63FAA, or a combination thereof, is increased in the sample from subject. In some embodiments of any of the methods herein, detecting the dysbiosis associated with type 2 diabetes comprises determining that Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, or a combination thereof, is decreased in the sample from subject.

In some embodiments of any of the methods herein, Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea C1B35, Roseburia inulinivorans A2-194, Ruminococcus sp. 5.1.39BFAA, Zinderia insecticola CARI, or a combination thereof, are depleted in the gastrointestinal tract of the subject.

Also provided herein are methods for treating a subject in need thereof that include decreasing a population of an increased bacterial strain in the subject, wherein the increased bacterial strain is selected from the group consisting of Coprobacillus sp. 29_1, Dorea longicatena 111-35, Eubacterium limosum KIST612, Hungatella hathewayi 12489931, Lachnospiraceae bacterium 5/1/63FAA, and a combination thereof.

In some embodiments of any of the methods herein, the subject has type 2 diabetes.

In some embodiments of any of the methods herein, decreasing the population of an increased bacterial strain comprises administering to the subject a bacteriophage. In some embodiments of any of the methods herein, decreasing the population of an increased bacterial strain comprises administering to the subject a composition comprising an effective amount of a bacterial strain selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

In some embodiments of any of the methods herein, the bacterial strain comprises Alistipes sp. HGB5. In some embodiments of any of the methods herein, the bacterial strain comprises Atopobium parvulum type strain (IPP 1246). In some embodiments of any of the methods herein, the bacterial strain comprises Bacteroides clarus DSM 22519. In some embodiments of any of the methods herein, the bacterial strain comprises Butyrivibrio crossotus T9-40 A. In some embodiments of any of the methods herein, the bacterial strain comprises Eubacterium hadrum B2-52. In some embodiments of any of the methods herein, the bacterial strain comprises Prevotella stercorea CB35. In some embodiments of any of the methods herein, the bacterial strain comprises Roseburia inulinivorans A2-194. In some embodiments of any of the methods herein, the bacterial strain comprises Ruminococcus sp. 5.1.39BFAA. In some embodiments of any of the methods herein, the bacterial strain comprises Zinderia insecticola CARI.

In some embodiments of any of the methods herein, the bacterial strain improves intestinal barrier function of the subject.

In some embodiments of any of the methods herein, the Alistipes sp. HGB5 has a 16S RNA gene that is at least 95% identical to SEQ ID NO:1. In some embodiments of any of the methods herein, the Alistipes sp. HGB5 has a 16S RNA gene that is at least 95% identical to SEQ ID NO:2.

In some embodiments of any of the methods herein, the Atopobium parvulum type strain (IPP 1246) has a 16S RNA gene that is at least 95% identical to SEQ ID NO:3. In some embodiments of any of the methods herein, the Atopobium parvulum type strain (IPP 1246) has a 16S RNA gene that is at least 95% identical to SEQ ID NO:4. In some embodiments of any of the methods herein, the Atopobium parvulum type strain (IPP 1246) has a 16S RNA gene that is at least 95% identical to SEQ ID NO:5. In some embodiments of any of the methods herein, the Atopobium parvulum type strain (IPP 1246) has a 16S RNA gene that is at least 95% identical to SEQ ID NO:6.

In some embodiments of any of the methods herein, the Bacteroides clarus DSM 22519 has a 16S RNA gene that is at least 95% identical to SEQ ID NO:7. In some embodiments of any of the methods herein, the Bacteroides clarus DSM 22519 has a 16S RNA gene that is at least 95% identical to SEQ ID NO:8. In some embodiments of any of the methods herein, the Bacteroides clarus DSM 22519 has a 16S RNA gene that is at least 95% identical to SEQ ID NO:9. In some embodiments of any of the methods herein, the Bacteroides clarus DSM 22519 has a 16S RNA gene that is at least 95% identical to SEQ ID NO:10. In some embodiments of any of the methods herein, the Bacteroides clarus DSM 22519 has a 16S RNA gene that is at least 95% identical to SEQ ID NO:11.

In some embodiments of any of the methods herein, the Butyrivibrio crossotus T9-40 A has a 16S RNA gene that is at least 95% identical to SEQ ID NO:12. In some embodiments of any of the methods herein, the Butyrivibrio crossotus T9-40 A has a 16S RNA gene that is at least 95% identical to SEQ ID NO:13. In some embodiments of any of the methods herein, the Butyrivibrio crossotus T9-40 A has a 16S RNA gene that is at least 95% identical to SEQ ID NO:14. In some embodiments of any of the methods herein, the Butyrivibrio crossotus T9-40 A has a 16S RNA gene that is at least 95% identical to SEQ ID NO:15.

In some embodiments of any of the methods herein, the Eubacterium hadrum B2-52 has a 16S RNA gene that is at least 95% identical to SEQ ID NO:16. In some embodiments of any of the methods herein, the Eubacterium hadrum B2-52 has a 16S RNA gene that is at least 95% identical to SEQ ID NO:17. In some embodiments of any of the methods herein, the Eubacterium hadrum B2-52 has a 16S RNA gene that is at least 95% identical to SEQ ID NO:18.

In some embodiments of any of the methods herein, the Prevotella stercorea CB35 has a 16S RNA gene that is at least 95% identical to SEQ ID NO:19.

In some embodiments of any of the methods herein, the Roseburia inulinivorans A2-194 has a 16S RNA gene that is at least 95% identical to SEQ ID NO:20.

In some embodiments of any of the methods herein, the Ruminococcus sp. 5.1.39BFAA has a 16S RNA gene that is at least 95% identical to SEQ ID NO:21. In some embodiments of any of the methods herein, the Ruminococcus sp. 5.1.39BFAA has a 16S RNA gene that is at least 95% identical to SEQ ID NO:22. In some embodiments of any of the methods herein, the Ruminococcus sp. 5.1.39BFAA has a 16S RNA gene that is at least 95% identical to SEQ ID NO:23. In some embodiments of any of the methods herein, the Ruminococcus sp. 5.1.39BFAA has a 16S RNA gene that is at least 95% identical to SEQ ID NO:24. In some embodiments of any of the methods herein, the Ruminococcus sp. 5.1.39BFAA has a 16S RNA gene that is at least 95% identical to SEQ ID NO:25. In some embodiments of any of the methods herein, the Ruminococcus sp. 5.1.39BFAA has a 16S RNA gene that is at least 95% identical to SEQ ID NO:26. In some embodiments of any of the methods herein, the Ruminococcus sp. 5.1.39BFAA has a 16S RNA gene that is at least 95% identical to SEQ ID NO:27. In some embodiments of any of the methods herein, the Ruminococcus sp. 5.1.39BFAA has a 16S RNA gene that is at least 95% identical to SEQ ID NO:28. In some embodiments of any of the methods herein, the Ruminococcus sp. 5.1.39BFAA has a 16S RNA gene that is at least 95% identical to SEQ ID NO:29.

In some embodiments of any of the methods herein, the Zinderia insecticola CARI has a 16S RNA gene that is at least 95% identical to SEQ ID NO:30. In some embodiments of any of the methods herein, the Zinderia insecticola CARI has a 16S RNA gene that is at least 95% identical to SEQ ID NO:31.

In some embodiments of any of the methods herein, the bacterial strain is selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and a combination thereof.

In some embodiments of any of the methods herein, the bacterial strain in the composition is viable. In some embodiments of any of the methods herein, the bacterial strain is lyophilized.

In some embodiments of any of the methods herein, the composition further comprises one or more cryopreservants.

In some embodiments of any of the methods herein, the therapeutically effective amount of the bacterial strain comprises at least about 1×10³ colony forming units (CFU) of the bacterial strain. In some embodiments of any of the methods herein, the therapeutically effective amount of the bacterial strain comprises about 1×10⁴ to about 1×10¹⁵ CFU of the bacterial strain. In some embodiments of any of the methods herein, the therapeutically effective amount of the bacterial strain comprises about 1×10⁶ to about 1×10¹⁰ CFU of the bacterial strain.

In some embodiments of any of the methods herein, the bacterial strain in the composition is non-viable. In some embodiments of any of the methods herein, the non-viable bacterial strain is heat-killed, irradiated, or lysed.

In some embodiments of any of the methods herein, the method comprises administering the composition to the subject once, twice, or three times per day.

In some embodiments of any of the methods herein, the composition is formulated for oral administration. In some embodiments of any of the methods herein, the composition is formulated as a tablet, a capsule, a powder, or a liquid. In some embodiments of any of the methods herein, the composition is formulated as a tablet. In some embodiments of any of the methods herein, the tablet is coated. In some embodiments of any of the methods herein, the coating comprises an enteric coating.

In some embodiments of any of the methods herein, the method further comprises administering another treatment of type 2 diabetes and/or other adjunct therapy to the subject.

In some embodiments of any of the methods herein, the composition comprising the bacterial strain treatment and the treatment for type 2 diabetes and/or adjunct therapy are administered simultaneously. In some embodiments of any of the methods herein, the composition comprising the bacterial strain treatment and the treatment for type 2 diabetes and/or adjunct therapy are administered sequentially. In some embodiments of any of the methods herein, the treatment for type 2 diabetes and/or adjunct therapy comprises a probiotic.

In some embodiments of any of the methods herein, the treatment for type 2 diabetes and/or adjunct therapy comprises a therapeutic agent. In some embodiments of any of the methods herein, the therapeutic agent comprises metformin, a sulfonylurea, a meglitinide, a thiazolidinedione, a DPP-4 inhibitor, a GLP-1 receptor agonist, a SGLT-2 inhibitor, insulin, or a combination thereof. In some embodiments of any of the methods herein, the therapeutic agent comprises metformin, glyburide, glipizide, glimepiride, repaglinide, nateglinide, rosiglitazone, pioglitazone, sitagliptin, saxagliptin, linagliptin, exenatide, liraglutide, semaglutide, canagliflozin, dapagliflozin, empagliflozin, insulin, or a combination thereof.

In some embodiments of any of the methods herein, the composition comprising the bacterial strain further comprises the therapeutic agent.

In some embodiments of any of the methods herein, the subject is a human.

Also provided herein are methods of early diagnosing type 2 diabetes in a subject that include: (a) early diagnosing type 2 diabetes in a subject having a sample that has:

(i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or (b) not early diagnosing type 2 diabetes in a subject that does not have: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

As used herein, the phrase an “effective amount” of a bacterial strain can refer to an amount of the bacterial strain sufficient enough to reduce or eliminate one or more symptoms of the disorder or in some cases, to effect a cure upon administration. Effective amounts of a bacterial strain will vary with the bacterial strain chosen, the particular condition or conditions being treated, the severity of the condition, the duration of the treatment, the specific components of the composition being used, and like factors. An “effective amount” can also refer to an amount of a combination of two or more bacterial strains or a combination of a bacterial strain and a therapeutic agent sufficient to reduce or eliminate one or more symptoms of the disorder or in some cases, to effect a cure upon administration. For example, an “effective amount” can refer to an amount of a combination of bacterial strains or a combination of a bacterial strain and another treatment (e.g., a therapeutic agent) when an additive or synergistic effect is observed with the combination compared to administration of the bacterial strain(s) and/or therapeutic agent(s) alone.

As used herein, “subject” or “patient” refers to any subject, particularly a mammalian subject such as a human, for whom diagnosis, prognosis, or therapy is desired.

As used herein, “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or improve a patient or subject's quality of life.

The term “preventing” as used herein means the prevention of the onset, recurrence, or spread, in whole or in part, of the disease or condition as described herein, or a symptom thereof.

The term “administration” or “administering” refers to a method of giving an amount of a bacterial strain, or a composition thereof, or other treatment of type 2 diabetes and/or adjunct therapy to a subject. The method of administration can vary depending on various factors, e.g., the components of the composition, the site of the disease, and the severity of the disease.

“Microbiome” refers to the collection of microorganisms and viruses and/or their genes from a given environment. For example, “microbiome” can refer to the collection of the microorganisms and viruses and/or their genes from the gastrointestinal tract of humans. “Microbiota” refers to the microorganisms in a specific environment.

“Dysbiosis” refers to a state of the microbiota or microbiome of the gut or other body area (e.g., mucosal or skin surfaces or any other microbiota niche) of a subject (i.e., the host) in which the diversity and/or function of the ecological network is disrupted, e.g., as compared to the state of the microbiota or microbiome of the gut or other body area in a control population. A control population can include individuals that meet one or more qualifications such as individuals that have not been diagnosed with a disease (e.g., the same disease as the subject); individuals that do not have a known genetic predisposition to a disease (e.g., the same disease as the subject); or individuals that do not have a known environmental predisposition to a disease (e.g., the same disease as the subject); or individuals that do not have a known predisposition that would prevent treatment of and/or recovery from a disease (e.g., the same disease as the subject). In some embodiments, the individuals in the control population meet one of the above control population qualifications. In some embodiments, the individuals in the control population meet two of the above control population qualifications. In some embodiments, the individuals in the control population meet three of the above control population qualifications. In some embodiments, the individuals in the control population meet four of the above control population qualifications. In some embodiments, the control population is homogenous with respect to at least one of the qualifications. Any disruption in the microbiota or microbiome of a subject (i.e., host) compared to the microbiota or microbiome of a control population can be considered a dysbiosis, even if such dysbiosis does not result in a detectable decrease in health of the subject. Dysbiosis in a subject may be unhealthy for the subject (e.g., result in a diseased state in the subject), it may be unhealthy for the subject under only certain conditions (e.g., result in diseased state under only certain conditions), or it may prevent the subject from becoming healthier (e.g., may prevent a subject from responding to treatment or recovering from a disease or disorder). Dysbiosis may be due to a decrease in diversity of the microbiota population composition (e.g., a depletion of one or more bacterial strains, an overgrowth of one or more bacterial strains, or a combination thereof), the overgrowth of one or more population of pathogens (e.g., a population of pathogenic bacteria) or pathobionts, the presence of and/or overgrowth of a symbiotic organism able to cause disease only when certain genetic and/or environmental conditions are present in a subject, or a shift to an ecological network that no longer provides a beneficial function to the host and therefore no longer promotes health.

As used herein the terms “microorganism” or “microbe” should be taken broadly. These terms are used interchangeably and include, but are not limited to, the two prokaryotic domains, Bacteria and Archaea, as well as eukaryotic fungi and protists. In some embodiments, the disclosure refers to a “bacterium” or a “microbe.” This characterization can refer to not only the identified taxonomic bacterial genera of the microbe, but also the identified taxonomic species, as well as the bacterial strains. A “strain” can include descendants of a single isolation in pure culture that is usually made up of a succession of cultures ultimately derived from an initial single colony. In some embodiments, a strain includes an isolate or a group of isolates that can be distinguished from other isolates of the same genus and species by phenotypic characteristics, genotypic characteristics, or both.

The term “relative abundance” as used herein, is the number or percentage of a microbe present in the gastrointestinal tract or any other microbiota niche of a subject, such as the ocular, placental, lung, cutaneous, urogenital, or oral microbiota niches, relative to the number or percentage of total microbes present in the gastrointestinal tract or the other microbiota niche of the subject. The relative abundance may also be determined for particular types of microbes such as bacteria, fungi, viruses, and/or protozoa, relative to the total number or percentage of bacteria, fungi, viruses, and/or protozoa present. Relative abundance can be determined by a number of methods readily known to the ordinarily skilled artisan, including, but not limited to, array or microarray hybridization, sequencing, quantitative PCR, and culturing and performance of colony forming unit (cfu, CFU) assays or plaque forming unit (pfu, PFU) assays performed on a sample from the gastrointestinal tract or other microbiota niche.

As used herein, terms such as “isolate” and “isolated” in reference to a microbe, are intended to mean that a microbe has been separated from at least one of the materials with which it is associated in a particular environment (for example gastrointestinal fluid, gastrointestinal tissue, human digestive fluid, human digestive tissue, etc.). Accordingly, an “isolated microbe” does not exist in its naturally occurring environment. In some embodiments, an isolated microbe, e.g., a bacterial strain, may exist as, for example, a biologically pure culture, or as spores (or other forms of the bacterial strain) in association with a pharmaceutically acceptable excipient suitable for human administration. In some embodiments, more than one microbe can be isolated. For example, “isolated microbes” can refer to a mixture of two or more microbes that have been separated from at least one of the materials with which they are associated in a particular environment.

In some embodiments, the isolated microbes exist as isolated and biologically pure cultures. As used herein, the term “biologically pure” refers to a composition comprising a species or strains of a microbe, wherein the composition is substantially free from the material from which the microbe was isolated or produced and from other microbes (e.g., other species or strains and other microbes of a different taxonomic classification). In some embodiments, “biologically pure” can refer to a composition that comprises a strain of a bacterial strain that is substantially free from the material from which the bacterial strain was isolated or produced and from other microbes, e.g., other strains of the same bacterial strain, other species of the same bacteria, and other bacteria and/or microbes of a different taxonomic classification). It will be appreciated by one of skill in the art, that an isolated and biologically pure culture of a particular microbe, denotes that said culture is substantially free (within scientific reason) of other living organisms and contains only the individual microbe in question. As used herein, “substantially free” means that a composition comprising a species or strain of a microbe is at least about 80%, about 85%, about 90%, about 91%, about 92%, about 93%, about 94%, about 95%, about 96%, about 97%, about 98%, about 99%, or more than about 99% free of the material from which the microbe was isolated or produced and from other microbes. In some embodiments, a biologically pure composition contains no other bacterial strain in quantities that can be detected by typical bacteriological techniques.

As used herein, the term “mutation” includes a natural or induced mutation comprising at least a single base or amino acid alteration in a DNA or protein sequence. For example, a mutation can include a base substitution, a deletion, an insertion, a transversion, or any other modification known to those skilled in the art, including a genetic modification introduced into a parent nucleotide or an amino acid sequence.

As used herein, “probiotic” refers to a substantially pure microbe (i.e., a single isolate) or a mixture of microbes, and may also include any additional components that can be administered to a subject (e.g., a human), for restoring or altering the microbiota or microbiome in the subject. In some embodiments, a probiotic or microbial inoculant composition can be administered with an agent to allow the microbe(s) to survive the environment of the gastrointestinal tract, i.e., to resist low pH and/or to grow in the gastrointestinal environment. In some embodiments, a composition as described herein includes a probiotic.

As used herein, “prebiotic” refers to an agent that increases the number and/or activity of one or more microbes. Such microbes can include microbes for restoring or altering the microbiota or microbiome of a subject. Non-limiting examples of a prebiotic include a fructooligosaccharide (e.g., oligofructose, inulin, or an inulin-type fructan), a galactooligosaccharide, an amino acid, an alcohol. See, for example, Ramirez-Farias et al. (2008. Br. J Nutr. 4:1-10) and Pool-Zobel and Sauer (2007. J Nutr. 137:2580-2584).

As used herein, a “live biotherapeutic product” or “LBP” refers to a biological product that: 1) contains live organisms, such as bacteria, and 2) is applicable to the prevention, treatment, and/or cure of a disease or condition of a subject.

A “combination” of two or more bacteria, e.g., bacterial strains, can refer to the physical co-existence of the bacteria, either in the same material or product. In some embodiments, a combination of two or more bacteria can include the temporal co-administration or co-localization of the two or more bacteria.

The terms “percent identity” or “identity” in the context of two or more nucleic acids or polypeptides, refers to the measurement of the similarity between the two or more sequences. The percent identity can be measured by any method known to one of skill in the art including using a sequence comparison software, an algorithm, and by visual inspection.

In general, the percent identity for two or more sequences (e.g., a nucleic acid or amino acid sequence), also referred to as the “percent sequence identity”, is calculated by determining the number of matched positions in the aligned nucleic acid or amino acid sequences, dividing the number of matched positions by the total number of aligned nucleotides or amino acids, respectively, and multiplying by 100. A matched position refers to a position in which identical nucleotides or amino acids occur at the same position in the aligned sequences. As an example, the total number of aligned nucleotides can refer to the minimum number of the 16S rRNA gene nucleotides that are necessary to align the second sequence, and does not include alignment (e.g., forced alignment) with non-16S rRNA gene sequences. The total number of aligned nucleotides may correspond to the entire 16S rRNA gene sequence or may correspond to fragments of the full-length 16S rRNA gene sequence.

Sequences can be aligned using an algorithm, for example, the algorithm as described by Altschul et al. (Nucleic Acids Res, 25:3389-3402, 1997) and incorporated into BLAST (basic local alignment search tool) programs, which are available at ncbi.nlm.nih.gov. BLAST searches or alignments can be performed to determine percent sequence identity between a 16S rRNA gene nucleic acid and any other sequence or portion thereof using the Altschul et al. algorithm. BLASTN can be used to align and compare the identity between nucleic acid sequences, while BLASTP can be used to align and compare the identity between amino acid sequences. When utilizing a BLAST program to calculate the percent identity between a 16S rRNA gene sequence and another sequence, the default parameters of the program are used.

Generally, a bacterial strain genomic sequence will contain multiple copies of 16S rRNA sequences. The 16S rRNA sequences can be used for making distinctions between species and strains. For example, if one or more of the 16S rRNA sequences shares less than 97% sequence identity from a reference sequence, then the two organisms from which the sequences were obtained can be of different species or strains.

The term “combination therapy” as used herein refers to a dosing regimen of one or more bacterial strains and one or more other treatments of type 2 diabetes and/or adjunct therapies, wherein the bacterial strain and other treatment (e.g., a therapeutic agent) are administered together or separately in a manner prescribed by a medical care taker or according to a regulatory agency. As can be appreciated in the art, a combination therapy can be administered to a patient for a period of time. In some embodiments, the period of time occurs following the administration of one or more of: a different bacterial strain, a different treatment/therapeutic agent, and a different combination of treatments/therapeutic agents to the subject. In some embodiments, the period of time occurs before the administration of one or more of: a different bacterial strain, a different treatment/therapeutic agent, and a different combination of therapeutic treatments/agents to the subject.

The term “fixed combination” means that one or more bacterial strains as described herein, or a composition thereof, and at least one other treatment and/or adjunct therapy (e.g., a prebiotic, a probiotic, metformin, a sulfonylurea, a meglitinide, a thiazolidinedione, a DPP-4 inhibitor, a GLP-1 receptor agonist, a SGLT-2 inhibitor, insulin, or a combination thereof), are both administered to a subject simultaneously in the form of a single composition or dosage.

The term “non-fixed combination” means that one or more bacterial strains as described herein, or a composition thereof, and at least one other treatment or adjunct therapy (e.g., a prebiotic, a probiotic, metformin, a sulfonylurea, a meglitinide, a thiazolidinedione, a DPP-4 inhibitor, a GLP-1 receptor agonist, a SGLT-2 inhibitor, insulin, or a combination thereof) are formulated as separate compositions or dosages such that they may be administered to a subject simultaneously or sequentially with variable intervening time limits. These also apply to cocktail therapies, e.g., the administration of three or more therapeutic agents.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a,” “an,” and “the” include plural references unless indicated otherwise. For example, “an” excipient includes one or more excipients. It is understood that aspects and variations of the invention described herein include “consisting of” and/or “consisting essentially of” aspects and variations.

The details of one or more embodiments of the invention are set forth in the accompanying drawings and the description below. Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is an exemplary schematic of the analysis.

FIG. 2 has plots showing the tallies of strains detected (after prevalence filtering). The top panel tallies the strains detected in increasing number of datasets. The middle panel shows the number of strains that were significantly differentially abundant in 1 or more datasets. The bottom panel shows the number of strains that exhibit concordance in direction of the log 2 fold change, in increasing proportions of datasets in which they were detected. Only strains detected in at least two datasets are included.

FIG. 3 is a plot showing that significant findings from isolated dataset analysis rarely exhibits concordance in the direction of shifts across multiple datasets. Each row represents a strain identified as significantly differentially abundant by at least one isolated analysis. Asterisk denotes dataset(s) where significant changes of a strain was observed. Cell color indicates direction of the log 2 fold change: reduced (decreased) (green) or enriched (increased) (pink) in dysbiosis and not detected (white). Cells are shaded dark if the direction of log 2 fold change is concordant across all datasets a strain was detected in, i.e., the strain is associated with homeostasis or dysbiosis only. See FIG. 8 for details on contrasts analyzed within each cohort of subjects.

FIG. 4 is a plot showing the distribution of effect sizes per dataset. WGS-NGS refers to whole genome shotgun sequencing via next generation sequencing (NGS).

FIG. 5 is a plot showing strains (dots) significantly differentially abundant in eubiotic or dysbiotic state by isolated-dataset analysis (grey squares) or MTMA (grey spheres) in type 2 diabetes. Strains (dots) are sized by the number of datasets in which they were detected and colored as follows: significant by isolated analysis only (dark green); MTMA only (purple); or both (blue). Solid lines connect MTMA results to strains and dashed lines connect isolated analysis results to strains. Thick and thin lines indicate significant and non-significant findings, respectively. Red and green lines indicate enrichment (increase) and reduction (decrease) in dysbiotic state, respectively. Annotation for strains described here are provided in FIG. 9.

FIG. 6 is a forest plot demonstrating distribution of log 2 fold changes and 95% confidence intervals for strains that were identified as significantly differentially abundant by MTMA. Circles and triangles indicate log 2 fold change estimated by isolated analysis and MTMA, respectively. Error bars in the forest plots correspond to the 95% confidence interval. Green and blue indicate significant and nonsignificant findings, respectively, and grey indicates cases where an adjusted p-value could not be imputed by the statistical test.

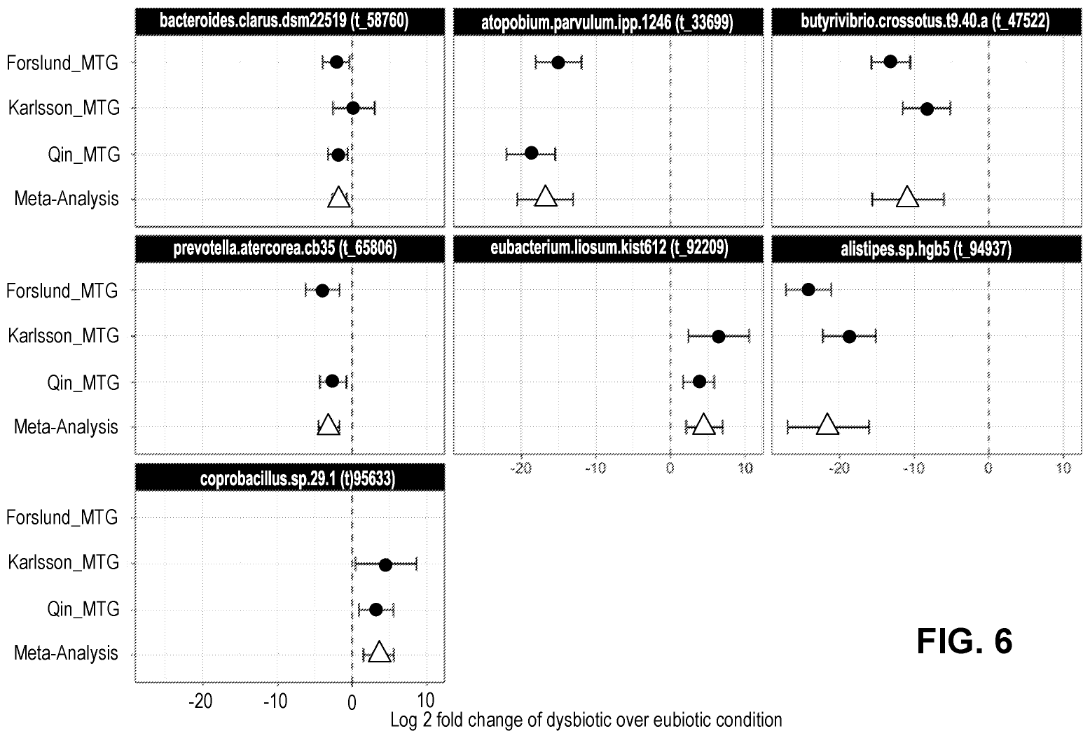

FIG. 7 is a plot of MTMA-derived adjusted p-values and log 2 fold changes. Data points are shaded according to the proportion of datasets in which the strain was detected. Significantly dysbiosis-associated strains plot in the upper left quadrant, whereas homeostasis-associated strains plot in the upper right quadrant. FIG. 9 provides strain names for strain identifiers indicated in the plot.

FIG. 8 is a table showing details on contrasts analyzed within each cohort of subjects.

FIG. 9 is a table showing the association between the strain identifiers and strain names.

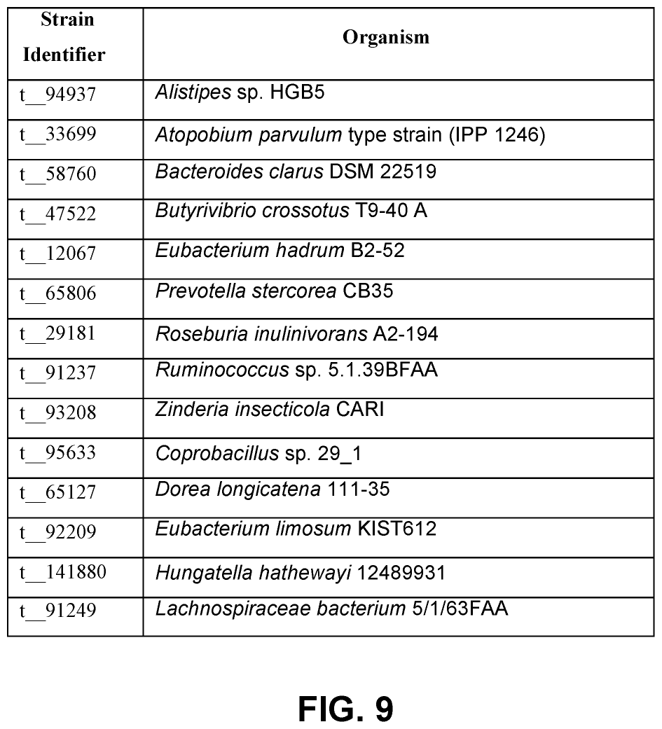

### DETAILED DESCRIPTION

This document provides compositions and methods for treating subjects in need thereof (e.g., subjects having type 2 diabetes) using one or more bacterial strains. When the pancreas is unable to produce enough insulin or when the body becomes resistant to insulin, Type 2 diabetes can develop. The exact etiology of type 2 diabetes is not known, but genetic and environmental factors, including being overweight and inactive, can be contributing factors

In some embodiments, methods for treating a subject in need thereof are provided herein. In some embodiments, one or more of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARI is reduced (decreased) in a sample (e.g., fecal sample or a biopsy sample such as an intestinal biopsy sample or a colorectal biopsy sample) from the subject in need thereof. For example, one or more of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea C1B35, Roseburia inulinivorans A2-194, Ruminococcus sp. 5.1.39BFAA, and Zinderia insecticola CARI is reduced (decreased) in the sample (e.g., fecal sample or a biopsy sample such as an intestinal biopsy sample or a colorectal biopsy sample) from the subject in need thereof compared to a control sample. Determining that one or more of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARI is reduced (decreased) in the sample from the subject in need thereof can comprise sequencing one or more nucleic acids from the bacteria. In some embodiments, the subject in need thereof has been diagnosed with type 2 diabetes. The methods provided herein can include administering to the subject a composition that includes an effective amount of a bacterial strain. In some embodiments, the bacterial strain can be selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof (e.g., any two, any three, any four, any five, any six, any seven, any eight, or nine of the bacterial strains). In some embodiments, the bacterial strain can be selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and a combination thereof (e.g., any two, any three, any four, any five, any six, any seven, or all eight of the bacterial strains).

In some embodiments, the bacterial strain in the composition comprises Alistipes sp. HGB5. A complete genomic sequence for Alistipes sp. HGB5 is available in the GenBank database as, e.g., Accession No. GCF_000183485. In some embodiments, the Alistipes sp. HGB5 included in a composition provided herein can have a genomic sequence with at least about 95% sequence identity to the genomic sequence published as GCF_000183485. For example, Alistipes sp. HGB5 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000183485. In some embodiments, Alistipes sp. HGB5 included in a composition provided herein has a 16S RNA gene that is at least 90% identical to one or both of SEQ ID NO:1 and SEQ ID NO:2 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:1 and SEQ ID NO:2).

In some embodiments, the bacterial strain in the composition comprises Atopobium parvulum type strain (IPP 1246). A complete genomic sequence for Atopobium parvulum type strain (IPP 1246) is available in the GenBank database as, e.g., Accession No. GCF_000024225. In some embodiments, the Atopobium parvulum type strain (IPP 1246) included in a composition provided herein can have a genomic sequence with at least about 95% sequence identity to the genomic sequence published as GCF_000024225. For example, Atopobium parvulum type strain (IPP 1246) included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000024225. In some embodiments, Atopobium parvulum type strain (IPP 1246) included in a composition provided herein can have a 16S RNA gene that is at least 90% identical to one or more (e.g., one, two, three, or all) of SEQ ID NO:3, SEQ ID NO:4, SEQ ID NO:5, and SEQ ID NO:6 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:3, SEQ ID NO:4, SEQ ID NO:5, and SEQ ID NO:6).

In some embodiments, the bacterial strain in the composition comprises Bacteroides clarus DSM 22519. A complete genomic sequence for Bacteroides clarus DSM 22519 is available in the GenBank database as, e.g., Accession No. GCF_000195615. In some embodiments, the Bacteroides clarus DSM 22519 included in a composition provided herein can have a genomic sequence with at least about 95% sequence identity to the genomic sequence published as GCF_000195615. For example, Bacteroides clarus DSM 22519 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000195615. In some embodiments, Bacteroides clarus DSM 22519 included in a composition provided herein has a 16S RNA gene that is at least 90% identical to one or more (e.g., one, two, three, four or all) of SEQ ID NO:7, SEQ ID NO: 8, SEQ ID NO: 9, SEQ ID NO: 10, and SEQ ID NO: 11 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four or all) of SEQ ID NO:7, SEQ ID NO: 8, SEQ ID NO: 9, SEQ ID NO: 10, and SEQ ID NO: 11).

In some embodiments, the bacterial strain in the composition comprises Butyrivibrio crossotus T9-40 A. A complete genomic sequence for Butyrivibrio crossotus T9-40 A is available in the GenBank database as, e.g., Accession No. GCF_000156015. In some embodiments, the Butyrivibrio crossotus T9-40 A strain included in a composition provided herein can have a genomic sequence with at least about 95% sequence identity to the genomic sequence published as GCF_000156015. For example, Butyrivibrio crossotus T9-40 A included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000156015. In some embodiments, Butyrivibrio crossotus T9-40 A included in a composition provided herein has a 16S RNA gene that is at least 90% identical to one or more (e.g., one, two, three, four or all) of SEQ ID NO:12, SEQ ID NO:13, SEQ ID NO:14, and SEQ ID NO:15 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four or all) of SEQ ID NO:12, SEQ ID NO:13, SEQ ID NO:14, and SEQ ID NO:15).

In some embodiments, the bacterial strain in the composition comprises Eubacterium hadrum B2-52. A complete genomic sequence for Eubacterium hadrum B2-52 is available in the GenBank database as, e.g., Accession No. GCF_000332875. In some embodiments, the Eubacterium hadrum B2-52 included in a composition provided herein can have a genomic sequence with at least about 95% sequence identity to the genomic sequence published as GCF_000332875. For example, Eubacterium hadrum B2-52 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000332875. In some embodiments, Eubacterium hadrum B2-52 included in a composition provided herein has a 16S RNA gene that is at least 90% identical to one or more (e.g., one, two, or all) of SEQ ID NO:16, SEQ ID NO:17, and SEQ ID NO:18 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, or all) of SEQ ID NO:16, SEQ ID NO:17, and SEQ ID NO:18).

In some embodiments, the bacterial strain in the composition comprises Prevotella stercorea CB35. A complete genomic sequence for Prevotella stercorea CB35 is available in the GenBank database as, e.g., Accession No. GCF_000235885. In some embodiments, the Prevotella stercorea CB35 included in a composition provided herein can have a genomic sequence with at least about 95% sequence identity to the genomic sequence published as GCF_000235885. For example, Prevotella stercorea CB35 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000235885. In some embodiments, Prevotella stercorea CB35 included in a composition provided herein has a 16S RNA gene that is at least 90% identical to SEQ ID NO:19 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:19).

In some embodiments, the bacterial strain in the composition comprises Roseburia inulinivorans A2-194. A complete genomic sequence for Roseburia inulinivorans A2-194 is available in the GenBank database as, e.g., Accession No. GCF_000174195. In some embodiments, the Roseburia inulinivorans A2-194 included in a composition provided herein can have a genomic sequence with at least about 95% sequence identity to the genomic sequence published as GCF_000174195. For example, Roseburia inulinivorans A2-194 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000174195. In some embodiments, Roseburia inulinivorans A2-194 included in a composition provided herein has a 16S RNA gene that is at least 90% identical to SEQ ID NO:20 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:20).

In some embodiments, the bacterial strain in the composition comprises Ruminococcus sp. 5.1.39BFAA. A complete genomic sequence for Ruminococcus sp. 5.1.39BFAA is available in the GenBank database as, e.g., Accession No. GCF_000159975. In some embodiments, the Ruminococcus sp. 5.1.39BFAA included in a composition provided herein can have a genomic sequence with at least about 95% sequence identity to the genomic sequence published as GCF_000159975. For example, Ruminococcus sp. 5.1.39BFAA included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000159975. In some embodiments, Ruminococcus sp. 5.1.39BFAA included in a composition provided herein has a 16S RNA gene that is at least 90% identical to one or more (e.g., one, two, three, four, five, six, seven, eight, or all) SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, or all) SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29).

In some embodiments, the bacterial strain in the composition comprises Zinderia insecticola CARI. A complete genomic sequence for Zinderia insecticola CARI is available in the GenBank database as, e.g., Accession No. GCF_000147015. In some embodiments, the Bacteroides clarus DSM 22519 strain included in a composition provided herein can have a genomic sequence with at least about 95% sequence identity to the genomic sequence published as GCF_000147015. For example, Zinderia insecticola CARI included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000147015. In some embodiments, strain Zinderia insecticola CARI included in a composition provided herein has a 16S RNA gene that is at least 90% identical to one or both of SEQ ID NO:30 and SEQ ID NO:31 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:30 and SEQ ID NO:31)

In some embodiments, the composition can include two or more bacterial strains selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARI. For example, the composition can include three or more, four or more, five or more, six or more, seven or more, eight or more, or all nine bacterial strains selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARL. Identifying characteristics of each strain are described above.

In some embodiments, a method can include detecting, in a sample from the subject, a dysbiosis associated with type 2 diabetes, e.g., before administering to the subject an effective amount of a bacterial strain or a composition containing the bacterial strain. In some embodiments, the sample is a fecal sample.

In some embodiments, detecting the dysbiosis associated with type 2 diabetes can include determining bacterial gene expression in the sample from the subject. (e.g., fecal sample). For example, the bacterial gene expression can be determined in the sample from the subject e.g., before administering to the subject an effective amount of a bacterial strain or a composition containing the bacterial strain and/or after administering to the subject an effective amount of a bacterial strain or a composition containing the bacterial strain. Determining the bacterial gene expression can include performing, for example, RNAseq and/or RT-qPCR. In some embodiments, detecting the dysbiosis associated with type 2 diabetes comprises determining bacterial composition in the sample from the subject (e.g., fecal sample). For example, the bacterial composition can be determined in a sample from the subject, e.g., before administering to the subject an effective amount of a bacterial strain or a composition containing the bacterial strain and/or after administering to the subject an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

In some embodiments, detecting the dysbiosis comprises determining that Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea C1B35, Roseburia inulinivorans A2-194, Ruminococcus sp. 5.1.39BFAA, Zinderia insecticola CARI, or a combination thereof, is reduced (decreased) in the sample from subject (e.g., reduced (decreased) in a fecal sample from the subject, or in the gastrointestinal tract of the subject).

In some embodiments, detecting the dysbiosis associated with type 2 diabetes comprises determining that Coprobacillus sp. 291, Dorea longicatena 111-35, Eubacterium limosum KIST612, Hungatella hathewayi 12489931, Lachnospiraceae bacterium 5/1/63FAA, or a combination thereof, is enriched (increased) in the sample from subject. In some embodiments, detecting the dysbiosis associated with type 2 diabetes comprises determining that Coprobacillus sp. 291, Dorea longicatena 111-35, Eubacterium limosum KIST612, Lachnospiraceae bacterium 5/1/63FAA, or a combination thereof, is enriched (increased) in the sample from subject.

In some embodiments, a method as provided herein can include decreasing a population of an enriched (increased) bacterial strain in a subject (e.g., a subject with type 2 diabetes). In some embodiments, detecting the decrease in the population of an enriched (increased) bacterial strain comprises determining the bacterial composition in a sample from the subject (e.g., a fecal sample). For example, the bacterial composition can be determined in a sample from the subject before administering to the subject an effective amount of a bacterial strain or a composition containing the bacterial strain and after administering to the subject an effective amount of a bacterial strain or a composition containing the bacterial strain. For example, the population of an enriched (increased) bacterial strain can be decreased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of a bacterial strain or a composition containing the bacterial strain to the subject compared to before administration to the subject of an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

In some embodiments, the enriched (increased) bacterial strain can be selected from the group consisting of Coprobacillus sp. 291, Dorea longicatena 111-35, Eubacterium limosum KIST612, Hungatella hathewayi 12489931, Lachnospiraceae bacterium 5/1/63FAA, and a combination thereof. In some embodiments, the enriched (increased) bacterial strain can be selected from the group consisting of Coprobacillus sp. 291, Dorea longicatena 111-35, Eubacterium limosum KIST612, Lachnospiraceae bacterium 5/1/63FAA, and a combination thereof.

In some embodiments, Coprobacillus sp. 29_1 has a 16S RNA gene that is at least 90% identical to SEQ ID NO:32 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:32).

A complete genomic sequence for Dorea longicatena 111-35 is available in the GenBank database as, e.g., Accession No. GCF_000154065. In some embodiments, the Dorea longicatena 111-35 strain can have a genomic sequence with at least about 95% sequence identity to the genomic sequence published as GCF_000154065. For example, Dorea longicatena 111-35 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000154065. In some embodiments, strain Dorea longicatena 111-35 can have a 16S RNA gene that is at least 90% identical to one or more (e.g., one, two, three, or all) of SEQ ID NO:33, SEQ ID NO:34, SEQ ID NO:35, and SEQ ID NO:36 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:33, SEQ ID NO:34, SEQ ID NO:35, and SEQ ID NO:36).

A complete genomic sequence for Eubacterium limosum KIST612 is available in the GenBank database as, e.g., Accession No. GCF_000152245. In some embodiments, Eubacterium limosum KIST612 can have a genomic sequence with at least about 95% sequence identity to the genomic sequence published as GCF_000152245. For example, Eubacterium limosum KIST612 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000152245. In some embodiments, Eubacterium limosum KIST612 can have a 16S RNA gene that is at least 90% identical to one or more (e.g., one, two, three, four, five, six, or all) of SEQ ID NO:37, SEQ ID NO:38, SEQ ID NO:39, SEQ ID NO:40, SEQ ID NO:41, SEQ ID NO:52, and SEQ ID NO:43 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:37, SEQ ID NO:38, SEQ ID NO:39, SEQ ID NO:40, SEQ ID NO:41, SEQ ID NO:52, and SEQ ID NO:43).

A complete genomic sequence for Hungatella hathewayi 12489931 is available in the GenBank database as, e.g., Accession No. GCF_000371445. In some embodiments, the Hungatella hathewayi 12489931 can have a genomic sequence with at least about 95% sequence identity to the genomic sequence published as GCF_000371445. For example, Hungatella hathewayi 12489931 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000371445. In some embodiments, Hungatella hathewayi 12489931 can have a 16S RNA gene that is at least 90% identical to SEQ ID NO:44 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:44).

In some embodiments, Lachnospiraceae bacterium 5/1/63FAA can have a 16S RNA gene that is at least 90% identical to one or more (e.g., one, two, or all) of SEQ ID NO:45, SEQ ID NO:46, and SEQ ID NO:47 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, or all) of SEQ ID NO:45, SEQ ID NO:46, and SEQ ID NO:47).

In some embodiments, decreasing the population of an enriched (increased) bacterial strain can include administering a bacteriophage to the subject. See, for example, Sabino et al. Aliment Pharmacol Ther. 51(1):53-63, 2020. In some embodiments, decreasing the population of an enriched (increased) bacterial strain can include administering to the subject a composition comprising an effective amount of a bacterial strain (e.g., a bacterial strain selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

In some embodiments, methods provided herein can include administering the composition that includes an effective amount of one or more bacterial strains to the subject at least once per day. For example, the composition can be administered two, three, four, or more times per day. In some embodiments, an effective amount of the bacterial strain is administered in one dose, e.g., once per day. In some embodiments, an effective amount of the bacterial strain is administered in more than one dose, e.g., more than once per day. In some embodiments, the method comprises administering the composition to the subject daily, every other day, every three days, or once a week.

In some embodiments, an effective amount of a bacterial strain (e.g., Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea C1B35, Roseburia inulinivorans A2-194, Ruminococcus sp. 5.1.39BFAA, Zinderia insecticola CARI, or a combination thereof) in a composition described herein can include at least about 1×10³ CFUs of the bacterial strain. For example, an effective amount of a bacterial strain can be at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFUs of the bacterial strain. In some embodiments, the therapeutically effective amount of a bacterial strain in a composition described herein comprises about 1×10³ to about 1×10¹⁵ CFUs of the bacterial strain (e.g., about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFUs of the bacterial strain).

In some embodiments, methods provided herein can include administering a composition comprising a bacterial strain as described herein in combination with one or more other treatments of type 2 diabetes and/or in combination with adjunct therapies such as a therapeutic agent. The composition comprising a bacterial strain and any other treatments and/or adjunct therapies can be administered together (e.g., in the same formulation), or the composition comprising the bacterial strain can be administered concurrently with, prior to, or subsequent to, the one or more other treatments or adjunct therapies.

In some embodiments, the treatment of type 2 diabetes and/or adjunct therapy administered in combination with a composition comprising a bacterial strain as described herein is a therapeutic agent such as metformin, a sulfonylurea, a meglitinide, a thiazolidinedione, a DPP-4 inhibitor, a GLP-1 receptor agonist, a SGLT-2 inhibitor, insulin, a probiotic, a prebiotic, or a combination thereof. In some embodiments, the additional therapeutic agent comprises metformin, glyburide, glipizide, glimepiride, repaglinide, nateglinide, rosiglitazone, pioglitazone, sitagliptin, saxagliptin, linagliptin, exenatide, liraglutide, semaglutide, canagliflozin, dapagliflozin, empagliflozin, insulin, or a combination thereof.

In some embodiments, a prebiotic and/or probiotic can be administered in combination with a composition comprising a bacterial strain as described herein. Non-limiting examples of a probiotic include one of more of Bifidobacteria (e.g., B. animalis, B. breve, B. lactis, B. longum, B. longum, or B. infantis), Lactobacillus (e.g., L. acidophilus, L. reuteri, L. bulgaricus, L. lactis, L. casei, L. rhamnosus, L. plantarum, L. paracasei, or L. delbreuckii/bulgaricus), Saccharomyces boulardii, E. coli Nissle 1917, and Streptococcus thermophiles. Non-limiting examples of a prebiotic include a fructooligosaccharide (e.g., oligofructose, inulin, or an inulin-type fructan), a galactooligosaccharide, an amino acid, or an alcohol. See, for example, Ramirez-Farias et al. (2008. Br. J Nutr. 4:1-10) and Pool-Zobel and Sauer (2007. J Nutr. 137:2580-2584).

In some embodiments, an effective amount of the treatment of diabetes and/or adjunct therapy is administered in combination with a composition comprising a bacterial strain as described herein.

In some embodiments, methods provided herein can include monitoring the subject after treatment with a composition described herein to determine if one or more symptoms have been alleviated, if the severity of one or more symptoms has been reduced, or if progression of the disease has been delayed or inhibited in the subject. There are numerous scores and clinical markers that can be utilized to assess the efficacy of administering a composition that includes bacterial strain as described herein in treating type 2 diabetes. Non-limiting examples of a score or clinical marker that can be utilized to assess the efficacy of administering a composition as described herein to a subject include blood glucose levels, blood insulin levels, blood C-peptide levels, blood active GLP-1 levels, and a hemoglobin A1c (HbA1c) test. In some embodiments, administration of a composition as described herein to a subject can decrease the subject's blood glucose levels, e.g., blood levels can decrease by at least about 10 mg/dl, e.g., at least 20 mg/dl, 30 mg/dl, 40 mg/dl, 50 mg/dl, 60 mg/dl, 70 mg/dl, 80 mg/dl, 90 mg/dl, 100 mg/dl or more. In some embodiments, an improvement in one or more of the above indexes or biomarkers after administering a bacterial strain, or a composition thereof, as described herein to the subject indicates treatment of type 2 diabetes.

In some embodiments, compositions provided herein can include one or more excipients and can be formulated for any of a number of delivery systems suitable for administration to a subject (e.g., probiotic or LBP delivery systems). Non-limiting examples of an excipient include a buffering agent, a diluent, a preservative, a stabilizer, a binding agent, a filler, a lubricant, a dispersion enhancer, a disintegrant, a lubricant, a disintegrant, a wetting agent, a glidant, a flavoring agent, a sweetener, and a coloring agent. For example, in some embodiments, tablets or capsules can be prepared by conventional means with excipients such as binding agents, fillers, lubricants, disintegrants, or wetting agents. Any of the compositions described herein can be administered to a subject to treat type 2 diabetes as described herein.

In some embodiments, a composition as described herein can be formulated for oral delivery. In some embodiments, the composition can be formulated as a tablet, a chewable tablet, a capsule, a stick pack, a powder, effervescent powder, or a liquid. In some embodiments, a composition can include coated beads that contain the bacterial strain. In some embodiments, a powder comprising the bacterial strain can be suspended or dissolved in a drinkable liquid such as water for administration. In some embodiments, the composition is a solid composition.

In some embodiments, a composition described herein can be formulated for various immediate and controlled release profiles of the bacterial strain. For example, a controlled release formulation can include a controlled release coating disposed over the bacterial strain. In some embodiments, the controlled release coating is an enteric coating, a semi-enteric coating, a delayed release coating, or a pulsed release coating. In some embodiments, a coating can be suitable if it provides an appropriate lag in active release (i.e., release of the bacterial strain). For example, in some embodiments, the composition can be formulated as a tablet that includes a coating (e.g., an enteric coating).

In some embodiments, the bacterial strain in the composition is a culture of a single strain of organism. In some embodiments, the composition comprises a bacterial strain that is isolated. In some embodiments, the bacterial strain is isolated and cultured in vitro to increase the number or concentration of the bacterial strain. Increasing the number or concentration of the bacterial strain can be useful, for example, to enhance the efficacy of a composition comprising the bacterial strain.

In some embodiments, an effective amount of the bacterial strain in a composition described herein comprises at least about 1×10³ CFU of the bacterial strain. For example, at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFU of the bacterial strain. In some embodiments, the effective amount of a bacterial strain in a composition described herein comprises about 1×10³ to about 1×10¹⁵ CFU of the bacterial strain. For example, about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFU of the bacterial strain.

In some embodiments, the composition can include one or more biologically pure strains (e.g., two or more, three or more, four or more, five or more, six or more, seven or more, eight or more, or nine bacterial strains). For example, the composition can include biologically pure Alistipes sp. HGB5, biologically pure Atopobium parvulum type strain (IPP 1246), biologically pure Bacteroides clarus DSM 22519, biologically pure Butyrivibrio crossotus T9-40 A, biologically pure Eubacterium hadrum B2-52, biologically pure Prevotella stercorea CB35, biologically pure Roseburia inulinivorans A2-194, biologically pure Ruminococcus sp. 5.1.39BFAA, biologically pure Zinderia insecticola CARI, or any combination thereof.

In some embodiments, the composition is a solid composition that includes at least 1×10³ CFU of a bacterial strain (e.g., a biologically pure strain) and one or more excipients. Identifying characteristics of suitable strains, including homology to 16S rRNA sequences are described above.

In some embodiments, each member of the same bacterial strain has a 16S rRNA gene sequence with at least about 90%, about 91%, about 92%, about 93%, about 94%, about 95%, about 96%, about 97%, about 98%, about 99%, about 99.5%, about 99.6%, about 99.7%, about 99.8%, or about 99.9% sequence identity to the 16S rRNA gene sequence of each other member of the same bacterial strain.

In some embodiments, a bacterial strain in a composition described herein is preserved. Methods for preserving bacterial strains can include lyophilization and cryopreservation, optionally in the presence of a protectant. Non-limiting examples of protectants include sucrose, inulin, and glycerol. In some embodiments, a composition can include a lyophilized or cryopreserved bacterial strain such as Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, or a combination thereof, and an optional protectant.

In some embodiments, wherein the bacterial strain is a combination of two or more of: Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea C1B35, Roseburia inulinivorans A2-194, Ruminococcus sp. 5.1.39BFAA, and Zinderia insecticola CARI, one or more of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARI are lyophilized or cryopreserved.

In some embodiments, the composition is a live bacterial product (LBP). In some embodiments, the bacterial strain in the composition is viable. The viable bacterial strain may be, for example, cryopreserved and/or lyophilized. In some embodiments, a composition for delivery of live bacterial strains (e.g., Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, or a combination thereof), can be formulated to maintain viability of the bacterial strain. In some embodiments, the composition comprises elements that protect the bacterial strain from the acidic environment of the stomach (e.g., an enteric coating).

In some embodiments, wherein the bacterial strain is a combination of two or more of: Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea C1B35, Roseburia inulinivorans A2-194, Ruminococcus sp. 5.1.39BFAA, and Zinderia insecticola CARI, one or more of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARI are viable.

In some embodiments, the bacterial strain in the composition can be non-viable. In some embodiments, the non-viable bacterial strain is heat-killed, irradiated, or lysed.

In some embodiments, wherein the bacterial strain is a combination of two or more of: Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea C1B35, Roseburia inulinivorans A2-194, Ruminococcus sp. 5.1.39BFAA, and Zinderia insecticola CARI, one or more of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and Zinderia insecticola CARI are are non-viable (e.g., heat-killed, irradiated, or lysed).

In some embodiments, the bacterial strain as described herein may be used in prophylactic applications. For example, in a prophylactic application, a bacterial strain or a composition described herein can be administered to a subject susceptible to, or otherwise at risk of, a particular disease in an amount that is sufficient to at least partially reduce the risk of developing a disease. One of ordinary skill in the art will appreciate that the precise amounts of the bacterial strain administered may depend on a number of subject specific factors such as the subject's state of health and/or weight.

Also provided herein are methods of identifying a subject as having or having an increased likelihood of developing type 2 diabetes that include: (a) identifying a subject having a sample that has: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, as having or having an increased likelihood of developing type 2 diabetes; or (b) identifying a subject having a sample that does not have: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as not having or not having an increased likelihood of developing type 2 diabetes.

Also provided herein are methods of diagnosing a subject as having type 2 diabetes that include: (a) identifying a subject having a sample that has: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as having type 2 diabetes; or (b) identifying a subject having a sample that does not have: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as not having type 2 diabetes.

Also provided herein are methods of treating type 2 diabetes in a subject that include: (a) administering a type 2 diabetes therapy to a subject determined to have a sample that has: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or (b) not administering a type 2 diabetes therapy to a subject determined not to have a sample that has: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

Also provided herein are methods for treating a subject in need thereof that include: (a) administering a composition comprising an effective amount of a bacterial species selected from the group consisting of Alistipes sp., Atopobium parvulum type, Bacteroides clarus, Butyrivibrio crossotus, Eubacterium hadrum, Prevotella stercorea, Roseburia inulinivorans, Ruminococcus sp., and Zinderia insecticola, as a monotherapy, or in conjunction with another type 2 diabetes therapy, to a subject determined to have a sample that has: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or (b) not adminsitering a composition comprising an effective amount of a bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as a monotherapy, or in conjunection with another type 2 diabetes therapy, to a subject determined not to have a sample that has: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola. In some embodiments, the subject has type 2 diabetes.

Some embodiments of any of the methods described herein include detecting the level of one or more bacterial species in the sample from the subject. In some embodiments, the level of Alistipes sp., Atopobium parvulum, Bacteroides clarus, Butyrivibrio crossotus, Eubacterium hadrum, Prevotella stercorea, Roseburia inulinivorans, Ruminococcus sp., or Zinderia insecticola is increased in comparison to the same bacterial species in a reference sample (control sample).

In some embodiments, the method comprises determining that the sample has: (i) an increased level of two or more of: Coprobacillus, Dorea longicatena, Eubacterium limosum, Hungatella hathewayi, and Lachnospiraceae bacterium; and/or (ii) a decreased level of two or more of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

In some embodiments, the method comprises determining that the sample has: (i) an increased level of three or more of: Coprobacillus, Dorea longicatena, Eubacterium limosum, Hungatella hathewayi, and Lachnospiraceae bacterium; and/or (ii) a decreased level of three or more of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

In some embodiments, the method comprises determining that the sample has: (i) an increased level of four or more of: Coprobacillus, Dorea longicatena, Eubacterium limosum, Hungatella hathewayi, and Lachnospiraceae bacterium; and/or (ii) a decreased level of four or more of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

Also provided herein are methods of early diagnosing type 2 diabetes in a subject, the method comprising: (a) early diagnosing type 2 diabetes in a subject having a sample that has: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or (b) not early diagnosing type 2 diabetes in a subject that does not have: (i) an increased level of one or more (e.g., two or more, three or more, four or more, or five) bacterial species selected from the group consisting of: Coprobacillus, Dorea longicatena, Eubacterium limosum, Hungatella hathewayi, and Lachnospiraceae bacterium; and/or (ii) a decreased level of one or more (e.g., two or more, three or more, four or more, five or more, six or more, seven or more, eight or more, or nine) bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

The invention will be further described in the following examples, which do not limit the scope of the invention described in the claims.

### EXAMPLES

### Example 1. Identification of Bacterial Strains Associated with Type 2 Diabetes

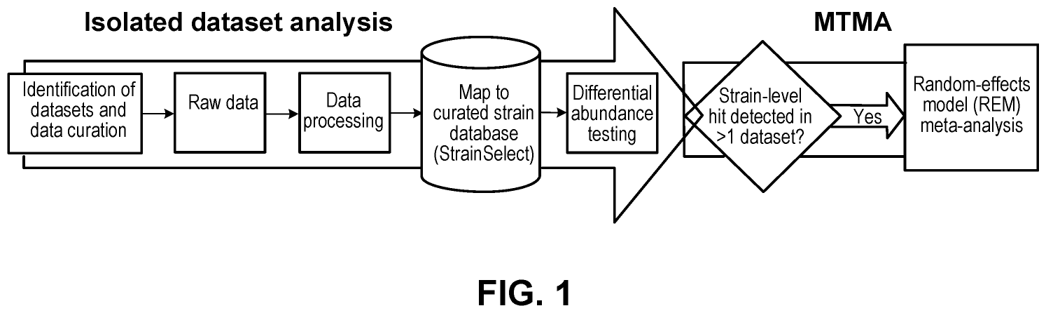

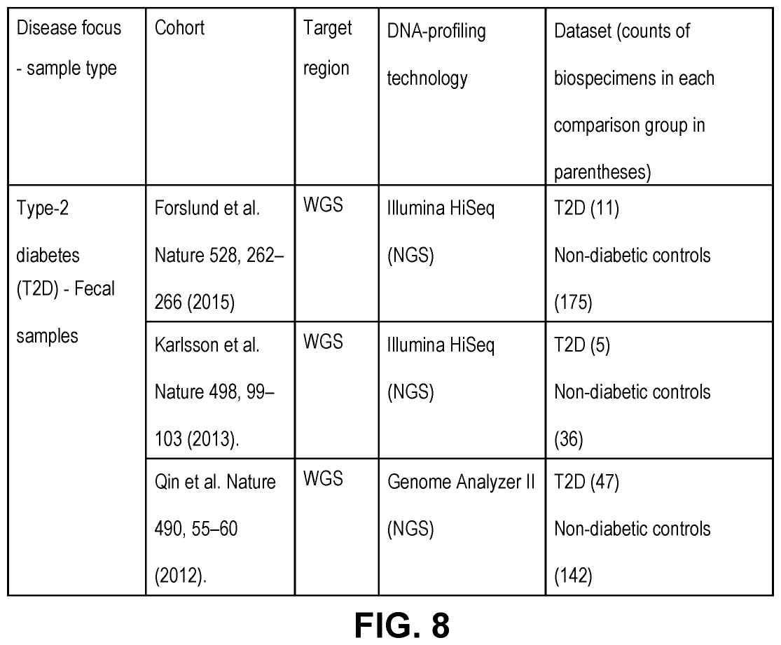

A multiple-technology meta-analysis (MTMA) approach was applied to datasets, which led to the identification of strains unique to type 2 diabetes. To overcome challenges of comparing clinical variables across datasets from multiple institutes, a controlled vocabulary of hierarchically organized terms and manually re-annotated metadata from public datasets using this vocabulary was created. FIG. 8 provides the details regarding the cohorts, datasets, and contrasts analyzed to identify differentially abundant (DA) taxa correlating with disease progression. Statistical analysis of each dataset was performed using the workflow described in FIG. 1. Strain-level annotation was achieved using StrainSelect, a database containing sequence information of bacterial and archaeal strains connected to genome identifiers, which facilitated comparative analysis of taxa abundances at a strain-level across datasets

### Methods

### Procurement of Raw Data and Metadata Curation

Fastq/Fasta files and metadata were procured from public repositories. Metadata stored with raw data, such as NCBI's RunInfo table associated with the SRA Run Selector, and/or metadata published in tables in the primary text or supplementary files of the publication, were retrieved and manually re-annotated using a controlled vocabulary of hierarchically organized terms. An in-house database was created to store all study-related data and facilitate appropriate metadata annotation of all datasets via manual curation. Clinical metadata was stored in this database as a series of label:value pairs attached to the biospecimen from which the data files were generated.

### Processing, Strain Annotation and Statistical Analyses of Raw Data

### Metagenomic Datasets (WGS-NGS)

Reads were processed with Trimmomatic (Bolger et al. Bioinformatics 30, 2114-2120 (2014)) to remove adapter sequences and low-quality ends (<Q20). Reads shorter than 35-bp following trimming were discarded. Contaminant sequences (e.g., sequencing primers) were removed using Bowtie (Langmead et al. Nat. Methods 9, 357-359 (2012)). Host sequences were removed via Kraken (Wood et al. Genome Biol. 15, R46 (2014)), which used exact alignments of raw shotgun sequences to k-mers derived from the human reference genome. Ribosomal RNA sequences from all three domains of life were identified and removed with SortMeRNA 2.0 (Kopylova et al. Bioinformatics 28, 3211-3217 (2012)). Raw, non-host reads were considered, while low quality reads and ribosomal RNA reads were ignored. MetaPhlAn2 (Metagenomic Phylogenetic Analysis, version 2.0) was used to taxonomically annotate the type-2 diabetes datasets. StrainSelect database version 2016 (SS16) was used to map genome assembly identifiers to strain-level annotations.

A 5% prevalence filter was applied to all bin (MetaPhlan2 generated taxon)-count tabulations. If a bin passed prevalence filtration yet went undetected in any of the samples within a particular contrast group, a pseudocount (the minimum relative abundance value observed in the dataset divided by 1000) was added to one random sample in that group. Wilcoxon-rank sum tests were applied to identify statistically significant differences in each bin, and p-values were Benjamini-Hochberg-corrected to adjust for false discovery. Significant results for isolated analysis were determined as adjusted p-values <0.05. Fold change, variance and standard error were calculated as described above.

### Multi-Technology Meta-Analysis

Log 2 fold change and standard errors pertaining to per-dataset statistical results in each disease area were integrated in MTMA using a Random effects model (REM), generated using the metafor R package. Only bins with strain-level annotations in each dataset, and only those strains observed in at least two datasets, post prevalence filtering, were retained for REM analysis. False discovery correction for REM generated p-values was achieved using the Benjamini-Hochberg method. Differences are deemed to be statistically significant at adjusted (Benjamini-Hochberg corrected) p-values <0.05 in both isolated dataset analysis and MTMA.

### Results

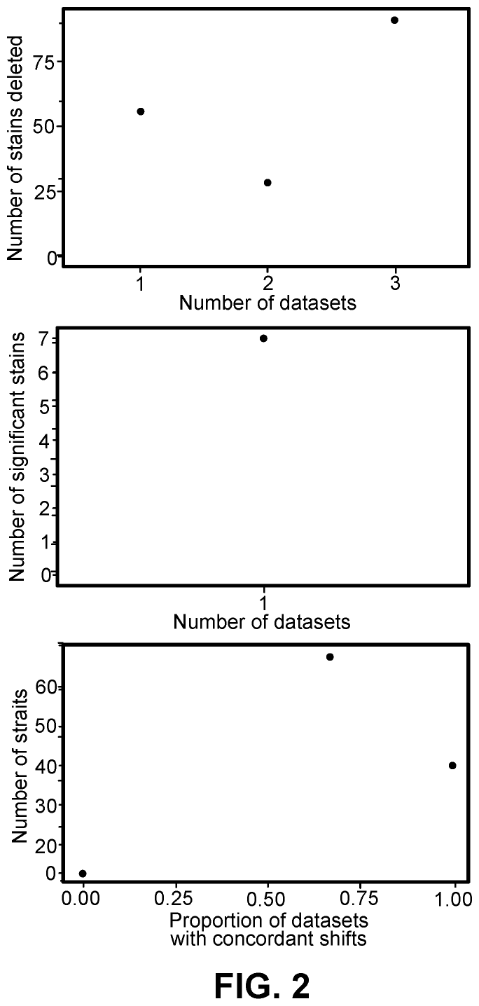

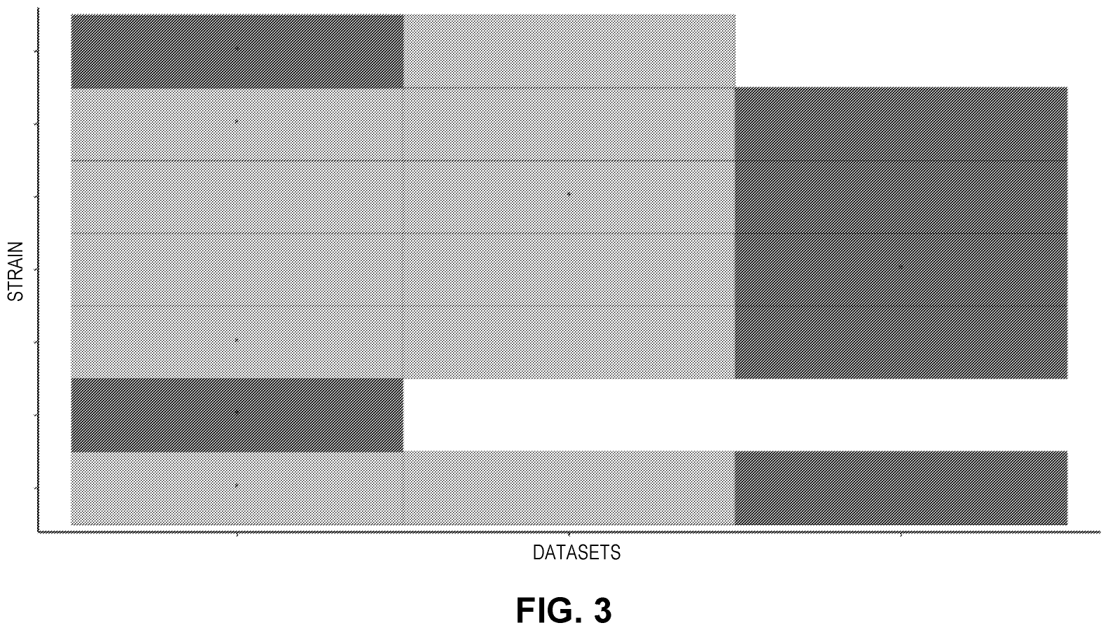

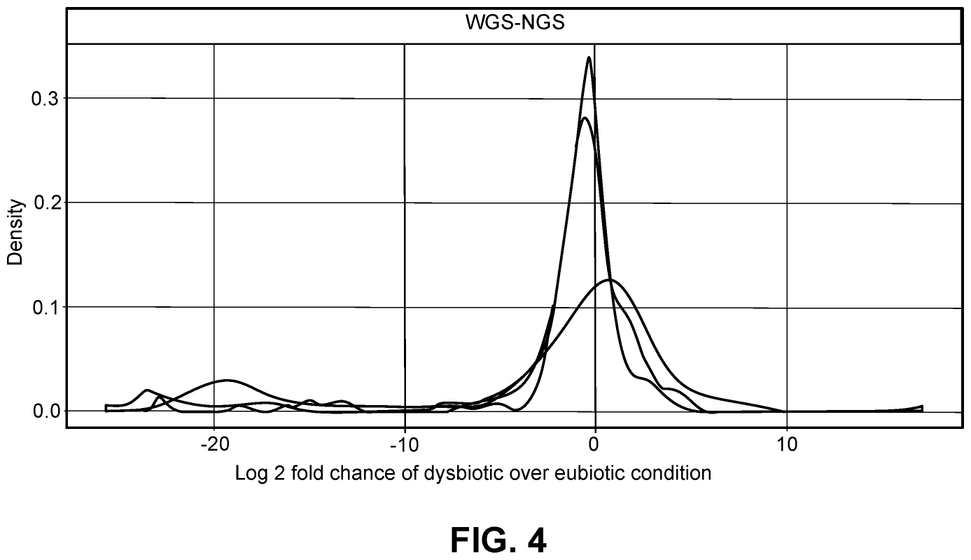

Identification of differentially abundant strains across cohorts from a simple comparison of isolated datasets was limited as only 51.7% of the strains were detected across all datasets (FIG. 2, top panel). Further, while isolated analysis identified 7 strains as significantly differentially abundant within a disease, these strain-disease associations were cohort-specific (FIG. 2, middle panel) and were not supported in trend, i.e., a strain being consistently associated with either homeostasis or dysbiosis, across other datasets (FIG. 3; light-shaded rows). Variation was also observed in the magnitude of differential abundance derived from the cohorts (log 2 fold change; FIG. 4).

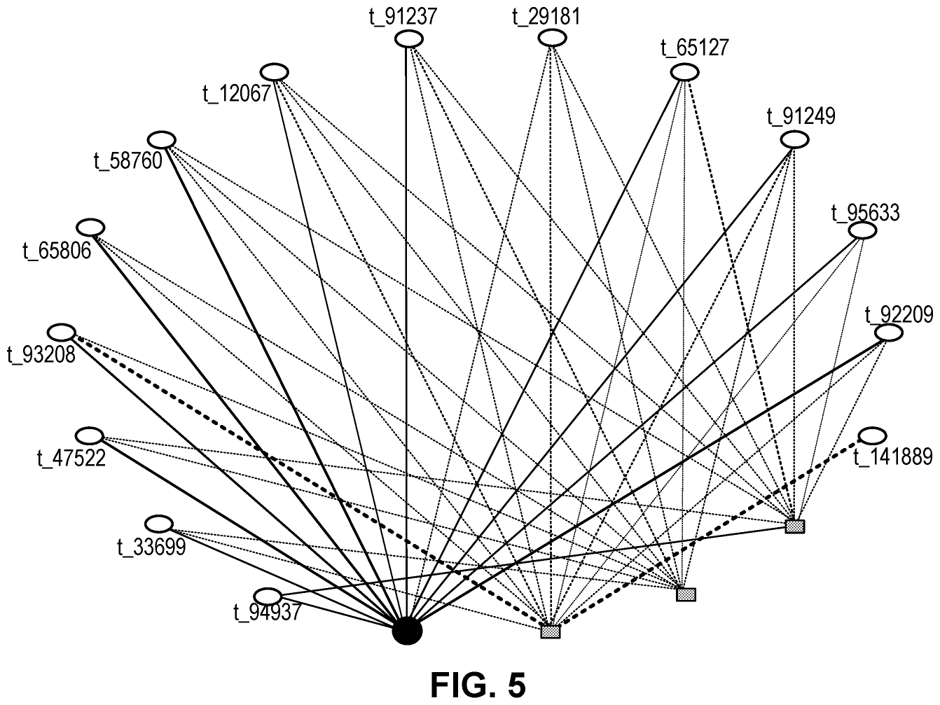

Strain-level results were integrated from isolated analyses in each disease via MTMA as described in FIG. 1. Significant associations were identified only when the direction of differential abundance of the strain was supported by multiple datasets (FIG. 5; blue strains connected to MTMA nodes via thick-solid lines). The associations in isolated analyses that were not supported in trend by other datasets were not significant by MTMA (FIG. 5; dark-green circles). Several strain-disease associations identified in MTMA were not identified in isolated analyses of the datasets (FIG. 5; purple dots). Thus, MTMA corroborates findings from isolated analysis if supported across datasets but eliminates if discordant, and MTMA identifies novel disease-strain associations that isolated analyses failed to detect.

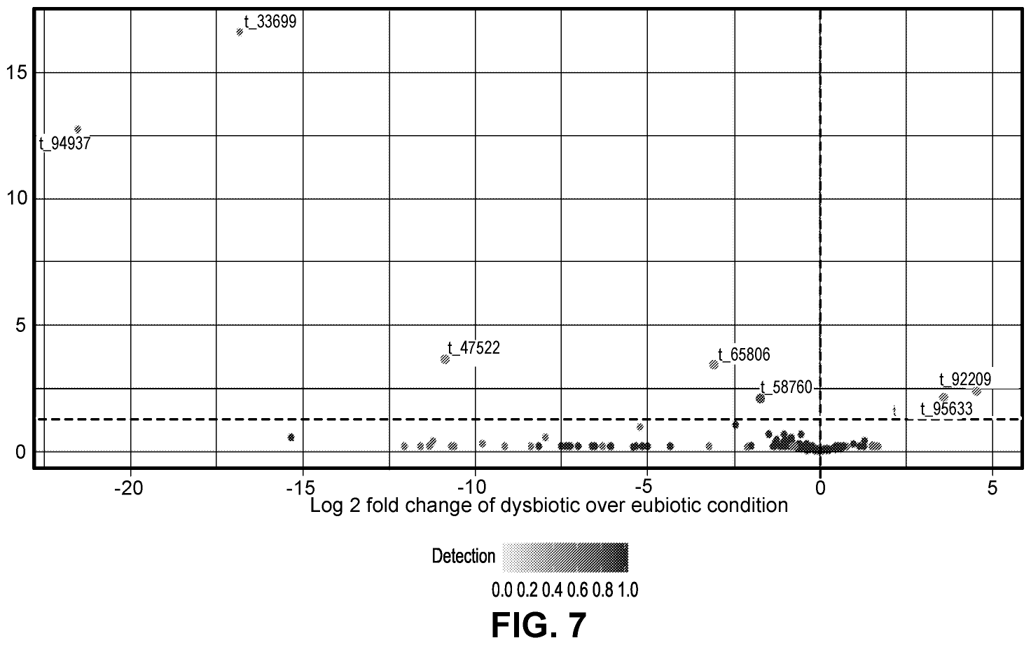

More strains were identified that were reduced (decreased) as opposed to enriched (increased) in disease indicating a correlation between these diseases and the lack of microbes associated with healthy subjects (FIG. 7). While the analysis of certain type 2 diabetes datasets confirmed the known reduction (decrease) of Akkermansia muciniphila in patients (Remely, et al. Endocr. Metab. Immune Disord. Drug Targets 16, 99-106 (2016)), this was not consistent across all cohorts. The lack of inference of a significant reduction (decrease) of A. muciniphila in type 2 diabetes using MTMA may stem from this association being cohort-specific or disease-heterogeneity in patient subsets.

MTMA can enable synthesis of existing knowledge of the microbiome, and the approach as shown in FIG. 1 can facilitate comparative analysis of taxa abundances at a strain-level across datasets generated with different DNA-profiling technologies. Harnessing the MTMA framework, with its ability to integrate datasets across DNA-profiling technologies and pinpoint specific strains, can allow for identification of robust microbiome modulators of disease by integrating the growing body of evidence on the role played by microbiome in disease.

### Other Embodiments

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

What is claimed is:

1. A method of identifying a subject as having or having an increased likelihood of developing type 2 diabetes, the method comprising:
(a) identifying a subject having a sample that has:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, as having or having an increased likelihood of developing type 2 diabetes; or
(b) identifying a subject having a sample that does not have:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as not having or not having an increased likelihood of developing type 2 diabetes.

2. A method of diagnosing a subject as having type 2 diabetes, the method comprising:
(a) identifying a subject having a sample that has:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as having type 2 diabetes; or
(b) identifying a subject having a sample that does not have:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as not having type 2 diabetes.

3. A method of treating type 2 diabetes in a subject, the method comprising:
(a) administering a type 2 diabetes therapy to a subject determined to have a sample that has:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or
(b) not administering a type 2 diabetes therapy to a subject determined not to have a sample that has:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

4. A method for treating a subject in need thereof, the method comprising:
(a) administering a composition comprising an effective amount of a bacterial species selected from the group consisting of Alistipes sp., Atopobium parvulum type, Bacteroides clarus, Butyrivibrio crossotus, Eubacterium hadrum, Prevotella stercorea, Roseburia inulinivorans, Ruminococcus sp., and Zinderia insecticola, as a monotherapy, or in conjunction with another type 2 diabetes therapy, to a subject determined to have a sample that has:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or
(b) not adminsitering a composition comprising an effective amount of a bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola, as a monotherapy, or in conjunection with another type 2 diabetes therapy, to a subject determined not to have a sample that has:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

5. The method of claim 4, wherein the subject has type 2 diabetes.

6. The method of any one of claims 1-5, wherein the method comprises detecting the level of one or more bacterial species in the sample from the subject.

7. The method of any one of claims 1-6, wherein the level of Alistipes sp., Atopobium parvulum, Bacteroides clarus, Butyrivibrio crossotus, Eubacterium hadrum, Prevotella stercorea, Roseburia inulinivorans, Ruminococcus sp., or Zinderia insecticola is increased in comparison to the same bacterial species in a reference sample.

8. The method of any one of claims 1-7, wherein the method comprises determining that the sample has:
(i) an increased level of two or more of: Coprobacillus, Dorea longicatena, Eubacterium limosum, Hungatella hathewayi, and Lachnospiraceae bacterium; and/or
(ii) a decreased level of two or more of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

9. The method of any one of claims 1-8, wherein the method comprises determining that the sample has:
(i) an increased level of three or more of: Coprobacillus, Dorea longicatena, Eubacterium limosum, Hungatella hathewayi, and Lachnospiraceae bacterium; and/or
(ii) a decreased level of three or more of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

10. The method of any one of claims 1-9, wherein the method comprises determining that the sample has:
(i) an increased level of four or more of: Coprobacillus, Dorea longicatena, Eubacterium limosum, Hungatella hathewayi, and Lachnospiraceae bacterium; and/or
(ii) a decreased level of four or more of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.

11. A method for treating type 2 diabetes in a subject, the method comprising administering to the subject a composition comprising an effective amount of a bacterial strain selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, and a combination thereof.

12. A method for treating type 2 diabetes in a subject, the method comprising:
(a) detecting a dysbiosis associated with type 2 diabetes in a sample from the subject; and
(b) administering to the subject a composition comprising an effective amount of a bacterial strain selected from the group consisting of Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, and a combination thereof.

13. The method of claim 12, wherein the sample is a fecal sample.

14. The method of any one of claim 12 or 13, wherein detecting the dysbiosis associated with type 2 diabetes comprises determining bacterial gene expression in the sample from the subject.

15. The method of any one of claims 12-14, wherein detecting the dysbiosis associated with type 2 diabetes comprises determining bacterial composition in the sample from the subject.

16. The method of claim any one of claims 12-15, wherein detecting the dysbiosis associated with type 2 diabetes comprises determining that Coprobacillus sp., Dorea longicatena, Eubacterium limosum, Hungatella hathewayi, Lachnospiraceae bacterium, or a combination thereof, is increased in the sample from subject.

17. The method of claim any one of claims 12-16, wherein detecting the dysbiosis associated with type 2 diabetes comprises determining that Alistipes sp., Atopobium parvulum type strain, Bacteroides clarus, Butyrivibrio crossotus, Eubacterium hadrum, Prevotella stercorea, Roseburia inulinivorans, Ruminococcus sp., Zinderia insecticola, or a combination thereof, is decreased in the sample from subject.

18. The method of claim 17, wherein Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, or a combination thereof, is decreased in the gastrointestinal tract of the subject.

19. A method for treating a subject in need thereof, the method comprising decreasing a population of an increased bacterial species in the subject, wherein the increased bacterial species is selected from the group consisting of Coprobacillus sp., Dorea longicatena, Eubacterium limosum, Hungatella hathewayi, Lachnospiraceae bacterium, and a combination thereof.

20. The method of claim 19, wherein the subject has type 2 diabetes.

21. The method of claim 19 or 20, wherein decreasing the population of an increased bacterial strain comprises administering to the subject a bacteriophage.

22. The method of any one of claims 19-21, wherein decreasing the population of an increased bacterial species comprises administering to the subject a composition comprising an effective amount of a bacterial species selected from the group consisting of Alistipes sp., Atopobium parvulum, Bacteroides clarus, Butyrivibrio crossotus, Eubacterium hadrum, Prevotella stercorea, Roseburia inulinivorans, Ruminococcus sp., Zinderia insecticola, and a combination thereof.

23. The method of any one of claims 1-18 and 22, wherein the bacterial species Alistipes sp. comprises the bacterial strain Alistipes sp. HGB5.

24. The method of any one of claims 1-18, 22, and 23, wherein the bacterial species Atopobium parvalum comprises the bacterial strain Atopobium parvulum type strain (IPP 1246).

25. The method of any one of claims 1-18 and 22-24, wherein the bacterial species Bacteroides clarus comprises the bacterial strain Bacteroides clarus DSM 22519.

26. The method of any one of claims 1-18 and 22-25, wherein the bacterial species Butyrivibrio crossotus comprises the bacterial strain Butyrivibrio crossotus T9-40 A.

27. The method of any one of claims 1-18 and 22-26, wherein the bacterial species Eubacterium hadrum comprises the bacterial strain Eubacterium hadrum B2-52.

28. The method of any one of claims 1-18 and 22-27, wherein the bacterial species Prevotella stercorea comprises the bacterial strain Prevotella stercorea CB35.

29. The method of any one of claims 1-18 and 22-28, wherein the bacterial species Roseburia inulinivorans comprises the bacterial strain Roseburia inulinivorans A2-194.

30. The method of any one of claims 1-18 and 22-29, wherein the bacterial species Ruminococcus sp. comprises the bacterial strain Ruminococcus sp. 5.1.39BFAA.

31. The method of any one of claims 1-18 and 22-30, wherein the bacterial species Zinderia insecticola comprises the bacterial strain Zinderia insecticola CARI.

32. The method of any one of claims 4-18 and 22-31, wherein the bacterial species improves intestinal barrier function of the subject.

33. The method of any one of claims 23-32, wherein the Alistipes sp. HGB5 has a 16S RNA gene that is at least 95% identical to SEQ ID NO:1.

34. The method of any one of claims 23-32, wherein the Alistipes sp. HGB5 has a 16S RNA gene that is at least 95% identical to SEQ ID NO:2.

35. The method of any one of claims 24-34, wherein the Atopobium parvulum type strain (IPP 1246) has a 16S RNA gene that is at least 95% identical to SEQ ID NO:3.

36. The method of any one of claims 24-34, wherein the Atopobium parvulum type strain (IPP 1246) has a 16S RNA gene that is at least 95% identical to SEQ ID NO:4.

37. The method of any one of claims 24-34, wherein the Atopobium parvulum type strain (IPP 1246) has a 16S RNA gene that is at least 95% identical to SEQ ID NO:5.

38. The method of any one of claims 24-34, wherein the Atopobium parvulum type strain (IPP 1246) has a 16S RNA gene that is at least 95% identical to SEQ ID NO:6.

39. The method of any one of claims 25-38, wherein the Bacteroides clarus DSM 22519 has a 16S RNA gene that is at least 95% identical to SEQ ID NO:7.

40. The method of any one of claims 25-38, wherein the Bacteroides clarus DSM 22519 has a 16S RNA gene that is at least 95% identical to SEQ ID NO:8.

41. The method of any one of claims 25-38, wherein the Bacteroides clarus DSM 22519 has a 16S RNA gene that is at least 95% identical to SEQ ID NO:9.

42. The method of any one of claims 25-38, wherein the Bacteroides clarus DSM 22519 has a 16S RNA gene that is at least 95% identical to SEQ ID NO:10.

43. The method of any one of claims 25-38, wherein the Bacteroides clarus DSM 22519 has a 16S RNA gene that is at least 95% identical to SEQ ID NO:11.

44. The method of any one of claims 26-43, wherein the Butyrivibrio crossotus T9-40 A has a 16S RNA gene that is at least 95% identical to SEQ ID NO:12.

45. The method of any one of claims 26-43, wherein the Butyrivibrio crossotus T9-40 A has a 16S RNA gene that is at least 95% identical to SEQ ID NO:13.

46. The method of any one of claims 26-43, wherein the Butyrivibrio crossotus T9-40 A has a 16S RNA gene that is at least 95% identical to SEQ ID NO:14.

47. The method of any one of claims 26-43, wherein the Butyrivibrio crossotus T9-40 A has a 16S RNA gene that is at least 95% identical to SEQ ID NO:15.

48. The method of any one of claims 27-47, wherein the Eubacterium hadrum B2-52 has a 16S RNA gene that is at least 95% identical to SEQ ID NO:16.

49. The method of any one of claims 27-47, wherein the Eubacterium hadrum B2-52 has a 16S RNA gene that is at least 95% identical to SEQ ID NO:17.

50. The method of any one of claims 27-47, wherein the Eubacterium hadrum B2-52 has a 16S RNA gene that is at least 95% identical to SEQ ID NO:18.

51. The method of any one of claims 28-50, wherein the Prevotella stercorea CB35 has a 16S RNA gene that is at least 95% identical to SEQ ID NO:19.

52. The method of any one of claims 29-51, wherein the Roseburia inulinivorans A2-194 has a 16S RNA gene that is at least 95% identical to SEQ ID NO:20.

53. The method of any one of claims 30-52, wherein the Ruminococcus sp. 5.1.39BFAA has a 16S RNA gene that is at least 95% identical to SEQ ID NO:21.

54. The method of any one of claims 30-52, wherein the Ruminococcus sp. 5.1.39BFAA has a 16S RNA gene that is at least 95% identical to SEQ ID NO:22.

55. The method of any one of claims 30-52, wherein the Ruminococcus sp. 5.1.39BFAA has a 16S RNA gene that is at least 95% identical to SEQ ID NO:23.

56. The method of any one of claims 30-52, wherein the Ruminococcus sp. 5.1.39BFAA has a 16S RNA gene that is at least 95% identical to SEQ ID NO:24.

57. The method of any one of claims 30-52, wherein the Ruminococcus sp. 5.1.39BFAA has a 16S RNA gene that is at least 95% identical to SEQ ID NO:25.

58. The method of any one of claims 30-52, wherein the Ruminococcus sp. 5.1.39BFAA has a 16S RNA gene that is at least 95% identical to SEQ ID NO:26.

59. The method of any one of claims 30-52, wherein the Ruminococcus sp. 5.1.39BFAA has a 16S RNA gene that is at least 95% identical to SEQ ID NO:27.

60. The method of any one of claims 30-52, wherein the Ruminococcus sp. 5.1.39BFAA has a 16S RNA gene that is at least 95% identical to SEQ ID NO:28.

61. The method of any one of claims 30-52, wherein the Ruminococcus sp. 5.1.39BFAA has a 16S RNA gene that is at least 95% identical to SEQ ID NO:29.

62. The method of any one of claims 31-61, wherein the Zinderia insecticola CARI has a 16S RNA gene that is at least 95% identical to SEQ ID NO:30.

63. The method of any one of claims 31-61, wherein the Zinderia insecticola CARI has a 16S RNA gene that is at least 95% identical to SEQ ID NO:31.

64. The method of any one of claims 4-18 and 23-63, wherein the bacterial species in the composition is viable.

65. The method of any one of claims 4-18 and 23-64, wherein the bacterial species is lyophilized.

66. The method of any one of claims 4-18 and 23-65, wherein the composition further comprises one or more cryopreservants.

67. The method of any one of claims 4-18 and 23-66, wherein the therapeutically effective amount of the bacterial species comprises at least about 1×10³ colony forming units (CFU) of the bacterial species.

68. The method of any one of claims 4-18 and 23-67, wherein the therapeutically effective amount of the bacterial species comprises about 1×10⁴ to about 1×10¹⁵ CFU of the bacterial species.

69. The method of any one of claims 4-18 and 23-68, wherein the therapeutically effective amount of the bacterial species comprises about 1×10⁶ to about 1×10¹⁰ CFU of the bacterial species.

70. The method of any one of claims 4-18 and 23-63, wherein the bacterial species in the composition is non-viable.

71. The method of claim 70, wherein the non-viable bacterial species is heat-killed, irradiated, or lysed.

72. The method of any one of claims 4-19 and 23-71, wherein the method comprises administering the composition to the subject once, twice, or three times per day.

73. The method of any one of claims 4-18 and 23-72, wherein the composition is formulated for oral administration.

74. The method of any one of claims 4-18 and 23-73, wherein the composition is formulated as a tablet, a capsule, a powder, or a liquid.

75. The method of any one of claims 4-18 and 23-73, wherein the composition is formulated as a tablet.

76. The method of claim 75, wherein the tablet is coated.

77. The method of claim 76, wherein the coating comprises an enteric coating.

78. The method of any one of claims 4-77, wherein the method further comprises administering another treatment of type 2 diabetes and/or other adjunct therapy to the subject.

79. The method of claim 78, wherein the composition comprising the bacterial species treatment and the treatment for type 2 diabetes and/or adjunct therapy are administered simultaneously.

80. The method of claim 78, wherein the composition comprising the bacterial species treatment and the treatment for type 2 diabetes and/or adjunct therapy are administered sequentially.

81. The method of any one of claims 78-80, wherein the treatment for type 2 diabetes and/or adjunct therapy comprises a probiotic.

82. The method of any one of claims 78-80, wherein the treatment for type 2 diabetes and/or adjunct therapy comprises a therapeutic agent.

83. The method of claim 82, wherein the therapeutic agent comprises metformin, a sulfonylurea, a meglitinide, a thiazolidinedione, a DPP-4 inhibitor, a GLP-1 receptor agonist, a SGLT-2 inhibitor, insulin, or a combination thereof.

84. The method of claim 83, wherein the therapeutic agent comprises metformin, glyburide, glipizide, glimepiride, repaglinide, nateglinide, rosiglitazone, pioglitazone, sitagliptin, saxagliptin, linagliptin, exenatide, liraglutide, semaglutide, canagliflozin, dapagliflozin, empagliflozin, insulin, or a combination thereof.

85. The method of any one of claims 82-84, wherein the composition comprising the bacterial species further comprises the therapeutic agent.

86. The method of any one of claims 1-85, wherein the subject is a human.

87. A method for treating a subject in need thereof, the method comprising administering to the subject a composition comprising an effective amount of a bacterial strain selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

88. The method of claim 87, wherein the subject has type 2 diabetes.

89. A method for treating type 2 diabetes in a subject, the method comprising administering to the subject a composition comprising an effective amount of a bacterial strain selected from the group consisting of: Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

90. A method for treating type 2 diabetes in a subject, the method comprising:
(a) detecting a dysbiosis associated with type 2 diabetes in a sample from the subject; and
(b) administering to the subject a composition comprising an effective amount of a bacterial strain selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

91. The method of claim 90, wherein the sample is a fecal sample.

92. The method of any one of claim 90 or 91, wherein detecting the dysbiosis associated with type 2 diabetes comprises determining bacterial gene expression in the sample from the subject.

93. The method of any one of claims 90-92, wherein detecting the dysbiosis associated with type 2 diabetes comprises determining bacterial composition in the sample from the subject.

94. The method of claim any one of claims 90-93, wherein detecting the dysbiosis associated with type 2 diabetes comprises determining that Coprobacillus sp. 291, Dorea longicatena 111-35, Eubacterium limosum KIST612, Hungatella hathewayi 12489931, Lachnospiraceae bacterium 5/1/63FAA, or a combination thereof, is increased in the sample from subject.

95. The method of claim any one of claims 90-94, wherein detecting the dysbiosis associated with type 2 diabetes comprises determining that Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, or a combination thereof, is decreased in the sample from subject.

96. The method of claim 95, wherein Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, or a combination thereof, are depleted in the gastrointestinal tract of the subject.

97. A method for treating a subject in need thereof, the method comprising decreasing a population of an increased bacterial strain in the subject, wherein the increased bacterial strain is selected from the group consisting of Coprobacillus sp. 29_1, Dorea longicatena 111-35, Eubacterium limosum KIST612, Hungatella hathewayi 12489931, Lachnospiraceae bacterium 5/1/63FAA, and a combination thereof.

98. The method of claim 97, wherein the subject has type 2 diabetes.

99. The method of claim 97 or 98, wherein decreasing the population of an increased bacterial strain comprises administering to the subject a bacteriophage.

100. The method of any one of claims 97-99, wherein decreasing the population of an increased bacterial strain comprises administering to the subject a composition comprising an effective amount of a bacterial strain selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, Zinderia insecticola CARI, and a combination thereof.

101. The method of any one of claims 87-96 and 100, wherein the bacterial strain comprises Alistipes sp. HGB5.

102. The method of any one of claims 87-96, 100, and 101, wherein the bacterial strain comprises Atopobium parvulum type strain (IPP 1246).

103. The method of any one of claims 87-96 and 100-102, wherein the bacterial strain comprises Bacteroides clarus DSM 22519.

104. The method of any one of claims 87-96 and 100-103, wherein the bacterial strain comprises Butyrivibrio crossotus T9-40 A.

105. The method of any one of claims 87-96 and 100-104, wherein the bacterial strain comprises Eubacterium hadrum B2-52.

106. The method of any one of claims 87-96 and 100-105, wherein the bacterial strain comprises Prevotella stercorea CB35.

107. The method of any one of claims 87-96 and 100-106, wherein the bacterial strain comprises Roseburia inulinivorans A2-194.

108. The method of any one of claims 87-96 and 100-107, wherein the bacterial strain comprises Ruminococcus sp. 5.1.39BFAA.

109. The method of any one of claims 87-96 and 100-108, wherein the bacterial strain comprises Zinderia insecticola CARI.

110. The method of any one of claims 87-96 and 100-109, wherein the bacterial strain improves intestinal barrier function of the subject.

111. The method of any one of claims 87-96 and 100-110, wherein the Alistipes sp. HGB5 has a 16S RNA gene that is at least 95% identical to SEQ ID NO:1.

112. The method of any one of claims 87-96 and 100-111, wherein the Alistipes sp. HGB5 has a 16S RNA gene that is at least 95% identical to SEQ ID NO:2.

113. The method of any one of claims 87-96 and 100-112, wherein the Atopobium parvulum type strain (IPP 1246) has a 16S RNA gene that is at least 95% identical to SEQ ID NO:3.

114. The method of any one of claims 87-96 and 100-113, wherein the Atopobium parvulum type strain (IPP 1246) has a 16S RNA gene that is at least 95% identical to SEQ ID NO:4.

115. The method of any one of claims 87-96 and 100-114, wherein the Atopobium parvulum type strain (IPP 1246) has a 16S RNA gene that is at least 95% identical to SEQ ID NO:5.

116. The method of any one of claims 87-96 and 100-115, wherein the Atopobium parvulum type strain (IPP 1246) has a 16S RNA gene that is at least 95% identical to SEQ ID NO:6.

117. The method of any one of claims 87-96 and 100-116, wherein the Bacteroides clarus DSM 22519 has a 16S RNA gene that is at least 95% identical to SEQ ID NO:7.

118. The method of any one of claims 87-96 and 100-117, wherein the Bacteroides clarus DSM 22519 has a 16S RNA gene that is at least 95% identical to SEQ ID NO:8.

119. The method of any one of claims 87-96 and 100-118, wherein the Bacteroides clarus DSM 22519 has a 16S RNA gene that is at least 95% identical to SEQ ID NO:9.

120. The method of any one of claims 87-96 and 100-119, wherein the Bacteroides clarus DSM 22519 has a 16S RNA gene that is at least 95% identical to SEQ ID NO:10.

121. The method of any one of claims 87-96 and 100-120, wherein the Bacteroides clarus DSM 22519 has a 16S RNA gene that is at least 95% identical to SEQ ID NO:11.

122. The method of any one of claims 87-96 and 100-121, wherein the Butyrivibrio crossotus T9-40 A has a 16S RNA gene that is at least 95% identical to SEQ ID NO:12.

123. The method of any one of claims 87-96 and 100-122, wherein the Butyrivibrio crossotus T9-40 A has a 16S RNA gene that is at least 95% identical to SEQ ID NO:13.

124. The method of any one of claims 87-96 and 100-123, wherein the Butyrivibrio crossotus T9-40 A has a 16S RNA gene that is at least 95% identical to SEQ ID NO:14.

125. The method of any one of claims 87-96 and 100-124, wherein the Butyrivibrio crossotus T9-40 A has a 16S RNA gene that is at least 95% identical to SEQ ID NO:15.

126. The method of any one of claims 87-96 and 100-125, wherein the Eubacterium hadrum B2-52 has a 16S RNA gene that is at least 95% identical to SEQ ID NO:16.

127. The method of any one of claims 87-96 and 100-126, wherein the Eubacterium hadrum B2-52 has a 16S RNA gene that is at least 95% identical to SEQ ID NO:17.

128. The method of any one of claims 87-96 and 100-127, wherein the Eubacterium hadrum B2-52 has a 16S RNA gene that is at least 95% identical to SEQ ID NO:18.

129. The method of any one of claims 87-96 and 100-128, wherein the Prevotella stercorea CB35 has a 16S RNA gene that is at least 95% identical to SEQ ID NO:19.

130. The method of any one of claims 87-96 and 100-129, wherein the Roseburia inulinivorans A2-194 has a 16S RNA gene that is at least 95% identical to SEQ ID NO:20.

131. The method of any one of claims 87-96 and 100-130, wherein the Ruminococcus sp. 5.1.39BFAA has a 16S RNA gene that is at least 95% identical to SEQ ID NO:21.

132. The method of any one of claims 87-96 and 100-131, wherein the Ruminococcus sp. 5.1.39BFAA has a 16S RNA gene that is at least 95% identical to SEQ ID NO:22.

133. The method of any one of claims 87-96 and 100-132, wherein the Ruminococcus sp. 5.1.39BFAA has a 16S RNA gene that is at least 95% identical to SEQ ID NO:23.

134. The method of any one of claims 87-96 and 100-133, wherein the Ruminococcus sp. 5.1.39BFAA has a 16S RNA gene that is at least 95% identical to SEQ ID NO:24.

135. The method of any one of claims 87-96 and 100-134, wherein the Ruminococcus sp. 5.1.39BFAA has a 16S RNA gene that is at least 95% identical to SEQ ID NO:25.

136. The method of any one of claims 87-96 and 100-135, wherein the Ruminococcus sp. 5.1.39BFAA has a 16S RNA gene that is at least 95% identical to SEQ ID NO:26.

137. The method of any one of claims 87-96 and 100-136, wherein the Ruminococcus sp. 5.1.39BFAA has a 16S RNA gene that is at least 95% identical to SEQ ID NO:27.

138. The method of any one of claims 87-96 and 100-137, wherein the Ruminococcus sp. 5.1.39BFAA has a 16S RNA gene that is at least 95% identical to SEQ ID NO:28.

139. The method of any one of claims 87-96 and 100-138, wherein the Ruminococcus sp. 5.1.39BFAA has a 16S RNA gene that is at least 95% identical to SEQ ID NO:29.

140. The method of any one of claims 87-96 and 100-139, wherein the Zinderia insecticola CARI has a 16S RNA gene that is at least 95% identical to SEQ ID NO:30.

141. The method of any one of claims 87-96 and 100-140, wherein the Zinderia insecticola CARI has a 16S RNA gene that is at least 95% identical to SEQ ID NO:31.

142. The method of any one of claims 87-96 and 100-141, wherein the bacterial strain is selected from the group consisting of Alistipes sp. HGB5, Atopobium parvulum type strain (IPP 1246), Bacteroides clarus DSM 22519, Butyrivibrio crossotus T9-40 A, Eubacterium hadrum B2-52, Prevotella stercorea CB35, Roseburia inulinivorans A2-194, Ruminococcus sp. 5.1.39BFAA, and a combination thereof.

143. The method of any one of claims 87-96 and 100-142, wherein the bacterial strain in the composition is viable.

144. The method of any one of claims 87-96 and 100-143, wherein the bacterial strain is lyophilized.

145. The method of any one of claims 87-96 and 100-144, wherein the composition further comprises one or more cryopreservants.

146. The method of any one of claims 87-96 and 100-145, wherein the therapeutically effective amount of the bacterial strain comprises at least about 1×10³ colony forming units (CFU) of the bacterial strain.

147. The method of any one of claims 87-96 and 100-146, wherein the therapeutically effective amount of the bacterial strain comprises about 1×10⁴ to about 1×10¹⁵ CFU of the bacterial strain.

148. The method of any one of claims 87-96 and 100-147, wherein the therapeutically effective amount of the bacterial strain comprises about 1×10⁶ to about 1×10¹⁰ CFU of the bacterial strain.

149. The method of any one of claims 87-96, and 100-148, wherein the bacterial strain in the composition is non-viable.

150. The method of claim 149, wherein the non-viable bacterial strain is heat-killed, irradiated, or lysed.

151. The method of any one of claims 87-96 and 100-150, wherein the method comprises administering the composition to the subject once, twice, or three times per day.

152. The method of any one of claims 87-96 and 100-151, wherein the composition is formulated for oral administration.

153. The method of any one of claims 87-96 and 100-152, wherein the composition is formulated as a tablet, a capsule, a powder, or a liquid.

154. The method of any one of claims 87-96 and 100-153, wherein the composition is formulated as a tablet.

155. The method of claim 154, wherein the tablet is coated.

156. The method of claim 155, wherein the coating comprises an enteric coating.

157. The method of any one of claims 87-156, wherein the method further comprises administering another treatment of type 2 diabetes and/or other adjunct therapy to the subject.

158. The method of claim 157, wherein the composition comprising the bacterial strain treatment and the treatment for type 2 diabetes and/or adjunct therapy are administered simultaneously.

159. The method of claim 157, wherein the composition comprising the bacterial strain treatment and the treatment for type 2 diabetes and/or adjunct therapy are administered sequentially.

160. The method of any one of claims 157-159, wherein the treatment for type 2 diabetes and/or adjunct therapy comprises a probiotic.

161. The method of any one of claims 157-160, wherein the treatment for type 2 diabetes and/or adjunct therapy comprises a therapeutic agent.

162. The method of claim 157-161, wherein the therapeutic agent comprises metformin, a sulfonylurea, a meglitinide, a thiazolidinedione, a DPP-4 inhibitor, a GLP-1 receptor agonist, a SGLT-2 inhibitor, insulin, or a combination thereof.

163. The method of claim 162, wherein the therapeutic agent comprises metformin, glyburide, glipizide, glimepiride, repaglinide, nateglinide, rosiglitazone, pioglitazone, sitagliptin, saxagliptin, linagliptin, exenatide, liraglutide, semaglutide, canagliflozin, dapagliflozin, empagliflozin, insulin, or a combination thereof.

164. The method of any one of claims 161-163, wherein the composition comprising the bacterial strain further comprises the therapeutic agent.

165. The method of any one of claims 87-164, wherein the subject is a human.

166. A method of early diagnosing type 2 diabetes in a subject, the method comprising:
(a) early diagnosing type 2 diabetes in a subject having a sample that has:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola; or
(b) not early diagnosing type 2 diabetes in a subject that does not have:
(i) an increased level of one or more bacterial species selected from the group consisting of: Coprobacillus, Dorea longicatena, Eubacterium limosum, Hungatella hathewayi, and Lachnospiraceae bacterium; and/or
(ii) a decreased level of one or more bacterial species selected from the group consisting of: Alistipes sp., Atopobium parvulum, Bacteroides clarus, Butyrivibrio crossotus, Eubacterium hadrum, Prevotella stercorea, Roseburia inulinivorans, Ruminococcus sp., and Zinderia insecticola.
